(19) United States
(12) Patent Application Publication
(10) Pub. No.: US 2004/0214798 A1
(43) Pub. Date: Oct. 28, 2004

(54) Nitroaryl phosphoramide compositions and methods for targeting and inhibiting undesirable cell growth or proliferation

(76) Inventor: Longqin Hu, Belle Mead, NJ (US)

Correspondence Address: Jane Massey Licata, Licata & Tyrrell P.C., 66 E. Main Street, Marlton, NJ, 08053, US

(21) Appl. No.: 10/421,000

(22) Filed: Apr. 22, 2003

(51) Int. Cl. A61K 31/675; A61K 31/66

(52) U.S. Cl. 514/85; 514/109; 514/89; 514/90; 514/91; 544/157; 544/337; 546/22; 548/413; 558/81

(57) ABSTRACT

The present invention relates to nitroaryl-substituted phosphoramide prodrug compounds and methods of producing the same for use in targeting and inhibiting undesirable cell growth or proliferation.

### BACKGROUND OF THE INVENTION

Many anticancer agents in clinical use are associated with serious side effects, such as gastrointestinal and bone marrow toxicity, due to the lack of selectivity for the target tumor cells.

Prodrugs have been designed to improve many of the undesirable physicochemical and biological properties of commonly used drugs (Pochopin, et al. (1995) 121:157-167; Oliyai and Stella (1993) Annu. Rev. Pharmacol. Toxicol. 32:521-544; Bundgaard, In: Design of Prodrugs, Elsevier, Amsterdam, 1985). Prodrug strategies have also been used in targeted drug delivery including antibody-directed enzyme prodrug therapy (ADEPT) and gene-directed enzyme prodrug therapy (GDEPT). In these approaches, an enzyme is delivered site-specifically by chemical conjugation or genetic fusion to a tumor-specific antibody or by enzyme gene delivery systems into tumor cells. The delivered enzyme then selectively activates the prodrug at the tumor cells. A number of these therapies are in development and have been reviewed (McNeish, et al. (1997) Adv. Drug Delivery Rev. 26:173-184; Niculescu-Duvaz and Springer (1997) Adv. Drug Delivery Rev. 22:151-172; Senter and Svensson (1996) Adv. Drug Delivery Rev. 22:341-349). One such enzyme is a bacterial nitroreductase from Escherichia coli B. This FMN-containing flavoprotein is capable of reducing certain aromatic nitro groups to the corresponding amines or hydroxylamines in the presence of a cofactor NADH or NADPH (Bridgewater, (1995) Eur. J. Canc. 31:2361-2370; Anlezark, et al. (1992) Biochem. Pharmacol. 44:2289-2295; Knox, et al. (1992) Biochem. Pharmacol. 44:2297-2301).

Improved, targeted agents which significantly inhibit undesirable cell growth or proliferation are needed. The present invention meets this long-felt need.

### SUMMARY OF THE INVENTION

One aspect of the present invention is a nitroaryl-substituted phosphoramide compound. The compound is of Formula I or Formula II:

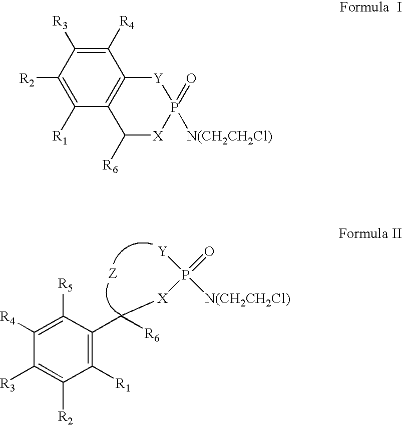

wherein at least one of R₁, R₃ or R₅ is a nitro group and the remaining substituents, R₁, R₂, R₃, R₄, and R₅, are independently a hydrogen, lower alkyl, amino, mono- or di-alkyl amino, alkanoyl amino, hydroxy, alkoxy, alkoxycarbonyl, carbamoyl, cyano, formyl, carboxyl or halogen group;

R₆ is a hydrogen, lower alkyl that is unsubstituted or substituted by free or alkylated amino, piperazinyl, piperidyl, pyrrolidinyl or morpholinyl, hydroxy, alkoxy, alkoxycarbonyl, carbamoyl, carboxyl or cyano group;

X and Y are each independently O, NH, NCH₂CH₂Cl or N(CH₂CH₂Cl)₂; and

Z is two separate hydrogens or a methylene, ethylene, or propylene that is unsubstituted or substituted by free or alkylated amino, piperazinyl, piperidyl, pyrrolidinyl or morpholinyl, hydroxy, alkoxy, alkoxycarbonyl, carbamoyl, or cyano.

Another aspect of the present invention is a method of producing a nitroaryl-substituted phosphoramide compound. The method involves a condensation reaction of a precursor alcohol, amino alcohol, diamine, or diol with bis(2-chloroethyl)phosphoramidic dichloride thereby producing a nitroaryl-substituted phosphoramide.

A further aspect of the present invention is a pharmaceutical composition containing a nitroaryl-substituted phosphoramide compound and a pharmaceutically acceptable carrier.

A still further aspect of the present invention is a method for inhibiting undesirable cell growth or proliferation. The method involves administering an effective amount of a pharmaceutical composition containing a nitroaryl-substituted phosphoramide compound and a pharmaceutically acceptable carrier so that undesirable cell growth or proliferation is inhibited, decreased or stabilized. In a preferred embodiment, the pharmaceutical composition is administered in combination with a reducing agent to activate the nitroaryl-substituted phosphoramide compound.

### DETAILED DESCRIPTION OF THE INVENTION

Cyclophosphamide (1) {2-[bis(2-chloroethyl)amino]-2H-1,3,2-oxazaphosphorinane 2-oxide} and derivatives thereof (U.S. Pat. No. 5,306,727) are clinically useful prodrugs which are activated by hepatic cytochrome P-450 enzyme (Zon (1982) Prog. Med. Chem. 19:205-246; Stec (1982) J. Organophosphorous Chem. 13:145-174; Borch and Millard (1987) J. Med. Chem. 30:427-431). Cytochrome P-450 oxidation converts cyclophosphamide to its corresponding 4-hydroxy derivative (2), which is ultimately converted to the cytotoxic alkylating species, phosphoramide mustard (5) (Scheme 1). Phosphoramide mustard formation is initiated by ring opening of 2 to produce aldophosphamide (3). The formation of 5 and 3 proceeds by general base-catalyzed β-elimination. Enzymes are not required for conversions following the initial hydroxylation in the liver (Borch and Millard (1987) supra). The aldehyde moiety in 3 serves as a substrate for aldehyde dehydrogenase and the corresponding carboxylic acid product is less prone to β-elimination. Aldehyde dehydrogenase is widely distributed in normal human tissues and has been found in cyclophosphamide-resistant tumor cells. However, most malignant tumor cells seem to have very little of this enzyme. Therefore, it is believed that the detoxication by aldehyde dehydrogenase might be responsible for its tumor selectivity as well as drug-resistance in resistant tumor cells (Hilton (1984) supra). The α,β-unsaturated aldehyde acrolein (4) is a potent electrophile and the causative agent of the bladder toxicity associated with cyclophosphamide (Cox (1979) Biochem. Pharmacol. 28:2045-2049).

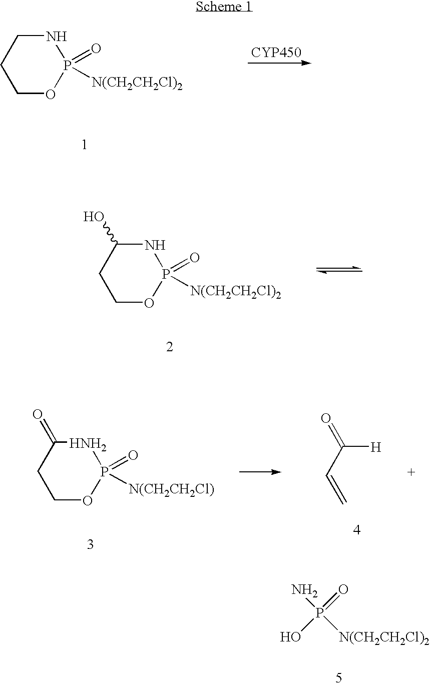

Solid tumors contain regions that are subject to chronic or transient deficiencies of blood flow leading to the development of chronic or acute hypoxia. Such oxygen deficiency often leads to resistance to ionizing radiation and to many chemotherapeutic drugs (Tercel, et al. (1996) J. Med. Chem. 39 (5): 1084-94). This common feature of solid tumors has led to novel chemotherapeutic approaches. Several examples of bioreductively-activated nitro compounds, quinones and aromatic N-oxides have been used as hypoxia-selective cytotoxins for development of selective anticancer prodrugs (Siim, et al. (1997) J. Med. Chem. 40 (9): 1381-90; Siim, et al. (1997) Cancer Res. 57 (14): 2922-8).

The methods and compositions provided herein relate to novel nitroaryl-substituted, cyclic and acyclic phosphoramide mustard derivatives for use in selectively targeting and inhibiting the growth or proliferation of undesirable cells.

Accordingly, one aspect of the present invention is a nitroaryl-substituted phosphoramide of Formulae I or II.

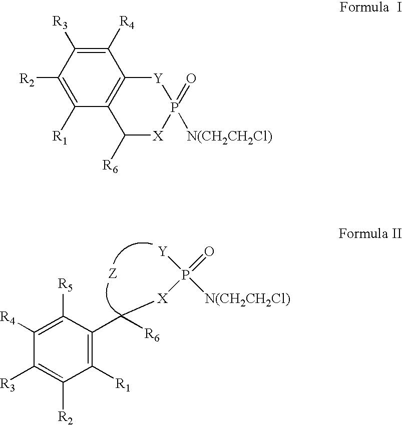

Wherein preferably at least one of R₁, R₃ or R₅ is a nitro group and most preferably R₃ is a nitro group and the remaining substituents, R₁, R₂, R₃, R₄, and R₅, may each independently be a hydrogen, lower alkyl, amino, mono- or di-alkyl amino, alkanoyl amino, hydroxy, alkoxy, alkoxycarbonyl, carbamoyl, cyano, formyl, carboxyl or halogen group.

In the nitroaryl-substituted phosphoramides of Formulae I and II, the R₆ moiety may be a hydrogen, lower alkyl that is unsubstituted or substituted by free or alkylated amino, piperazinyl, piperidyl, pyrrolidinyl or morpholinyl, hydroxy, alkoxy, alkoxycarbonyl, carbamoyl, carboxyl, cyano group or other suitable group which modifies the physicochemical property of the nitroaryl-substituted phosphoramide.

Preferably the X and Y moieties of Formulae I and II are each independently O, NH, NCH₂CH₂Cl or N(CH₂CH₂Cl)₂, and most preferably X is O and Y is NH, NCH₂CH₂Cl or N(CH₂CH₂Cl) 2.

In the nitroaryl-substituted phosphoramides of Formulae I and II, the Z moiety may be two separate hydrogens, representing an acyclic phosphoramide mustard; or methylene, ethylene, or propylene representing a 5, 6, or 7-member cyclophosphamide that is unsubstituted or substituted by free or alkylated amino, piperazinyl, piperidyl, pyrrolidinyl or morpholinyl, hydroxy, alkoxy, alkoxycarbonyl, carbamoyl, or cyano group.

In Formulae I and II, a lower alkyl is defined as having 1 to 6 carbon atoms, such as methyl, ethyl, propyl, isopropyl, butyl, isobutyl, sec-butyl, tert-butyl, pentyl, neopentyl, hexyl, and the like. Alkoxy refers to the group alkyl-O—. Preferred alkoxy groups include, for example, methoxy, ethoxy, n-propoxy, iso-propoxy, n-butoxy, tert-butoxy, sec-butoxy, n-pentoxy, n-hexoxy, 1,2-dimethylbutoxy, and the like. Halo or halogen refers to fluoro, chloro, bromo and iodo.

It is contemplated that chiral centers involving carbon or phosphorus present in the compounds of the Formulae I and II may independently of one another have R or S configurations. Compositions of Formulae I and II may contain pure enantiomers or pure diastereomers or mixtures of enantiomers, for example in the form of racemates, or mixtures of diastereomers. Mixtures of two or more stereoisomers of Formulae I or II are further contemplated with varying ratios of stereoisomers in the mixtures. Compositions of Formulae I or II may also contain trans- or cis-isomers including pure cis-isomers, pure trans-isomers or cis/trans-isomer mixtures with varying ratios of each isomer. When a composition containing a pure compound is desired, diastereomers (e.g., cis/trans-isomers) may be separated into the individual isomers (e.g, by chromatography) or racemates (e.g., separated using standard methods such as chromatography on chiral phases or resolution by crystallization of diastereomeric salts obtained with optically active acids or bases). Stereochemically uniform compositions of Formulae I or II may also be obtained by employing stereochemically uniform reactants or by using stereoselective reactions.

Salts of compounds of Formulae I or II may be obtained using methods well-known to those skilled in the art. For example, a salt may be obtained by combining a compound of the present invention with an inorganic or organic acid or base in a solvent or diluent, or from other salts by cation exchange or anion exchange. Salt-forming groups in a compound of Formulae I and II are groups or radicals having basic or acidic properties. Compounds having at least one basic group or at least one basic radical such as a free amino group, a pyrazinyl radical or a pyridyl radical, may form acid addition salts with, for example, inorganic acids such as hydrochloric acid, sulfuric acid, a phosphoric acid, or with suitable organic carboxylic or sulfonic acids. Suitable organic carboxylic or sulfonic acids may include aliphatic mono- or di-carboxylic acids (e.g., trifluoroacetic acid, acetic acid, propionic acid, glycolic acid, succinic acid, maleic acid, fumaric acid, hydroxymaleic acid, malic acid, tartaric acid, citric acid, oxalic acid); amino acids (e.g., arginine, lysine); aromatic carboxylic acids (e.g., benzoic acid, 2-phenoxy-benzoic acid, 2-acetoxy-benzoic acid, salicylic acid, 4-aminosalicylic acid); aromatic aliphatic carboxylic acids (e.g., mandelic acid, cinnamic acid); heteroaromatic carboxylic acids (e.g., nicotinic acid, isonicotinic acid); aliphatic sulfonic acids (e.g., methane-, ethane- or 2-hydroxyethane-sulfonic acid) or aromatic sulfonic acids (e.g., benzene-, p-toluene- or naphthalene-2-sulfonic acid). When several basic groups are present, mono- or poly-acid addition salts may be formed. Compounds of Formulae I and II having acidic groups, e.g., a free carboxy group in the radical R₆, may form metal or ammonium salts such as alkali metal or alkaline earth metal salts (e.g., sodium, potassium, magnesium or calcium salts) or ammonium salts with ammonia or suitable organic amines such as tertiary monoamines (e.g., triethylamine or tri-(2-hydroxyethyl)-amine), or heterocyclic bases (e.g., N-ethyl-piperidine or N,N′-dimethylpiperazine).

In the syntheses, purification and identification of the compounds of the present invention, the compounds are typically present in free and salt form, therefore as used herein, a free compound should be understood as including the corresponding salts.

Another aspect of the present invention includes methods of producing a nitroaryl-substituted phosphoramide compound of Formulae I or II. In general, the compounds of Formulae I and II may be prepared by condensation of a precursor alcohol, amino alcohol, diamine, or diol with bis(2-chloroethyl)phosphoramidic dichloride. When producing compounds of Formulae I or II it may be advantageous or necessary to introduce certain functional groups to avoid undesired reactions or side reactions in the respective synthesis step. These functional groups may include precursor groups which are later converted into the desired functional groups, or may be used to temporarily block a desired functional group by a protective group strategy suited to the synthesis. Such strategies are well-known to those skilled in the art (see, for example, Greene and Wuts, Protective Groups In Organic Synthesis, Wiley, 1999). Exemplary precursor or protective groups include, but are not limited to acyl or carbamoyl groups and azido groups which may be converted into an amino or hydroxy group via either hydrolysis or reduction.

One embodiment of the present invention is a method of producing a nitroaryl-fused cyclophosphamide compound of Formula I as depicted in Scheme 2, wherein Q is an amino or hydroxy protective group such as an acetyl or other lower alkanoyl, or alkoxycarbonyl group (e.g., tert-butyloxycarbonyl, fluorenylmethoxycarbonyl, benzyloxycarbonyl).

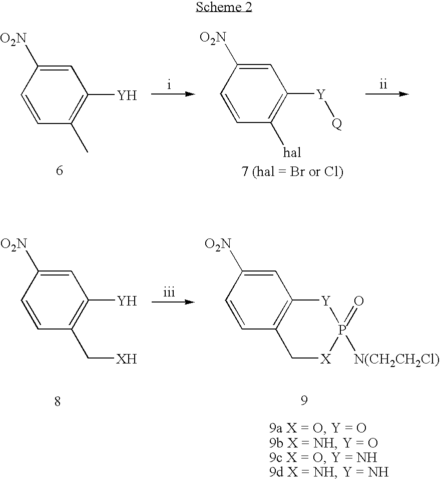

In general, the nitroaryl-fused cyclophosphamide 9 may be prepared according to the following steps of Scheme 2:

i) protecting the amino or hydroxyl group of a nitrophenol (6) using Ac₂O or another suitable anhydride and halogenating using NBS or another suitable reagent in the presence of light or a peroxide radical initiator; ii) converting the benzyl halide (7) to a primary amine, using the Gabriel synthesis, or an alcohol via hydrolysis; and iii) condensing with bis(2-chloroethyl)phosphoramidic dichloride in the presence of a base such as triethylamine.

Representative compounds of Formula I which may be produced in accordance with Scheme 2 include 7-nitro-2-[bis(2-chloroethyl)amino]-1,3,2-benzodioxaphosphorinane-2-oxide (9a); 7-nitro-2-[bis(2-chloroethyl)amino]-1,3,2-benzoxazaphos phorinane-2-oxide (9b); 7-nitro-2-[bis(2-chloroethyl)amino]-3,1,2-benzoxazaphosphorinane-2-oxide (9c); and 7-nitro-2-[bis(2-chloroethyl)amino]-1,3,2-benzodiazaphosphorinane-2-oxide (9d).

Another embodiment of the present invention is a method of producing a nitroaryl-substituted cyclophosphamide compound of Formula II as depicted in Scheme 3.

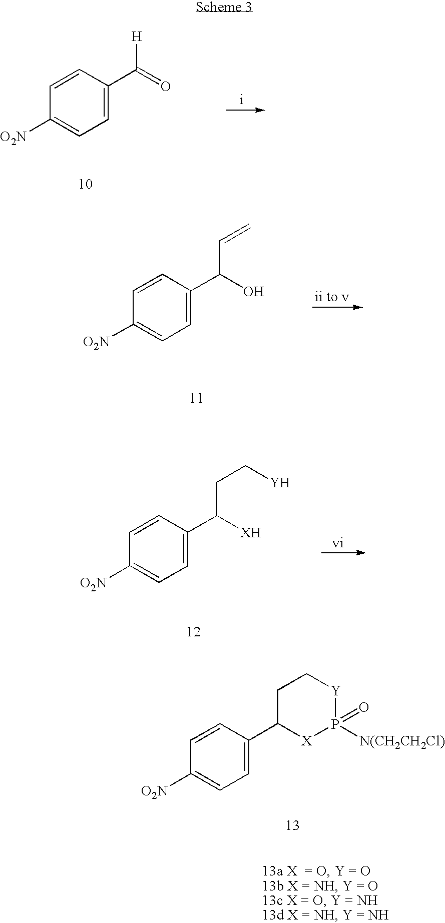

2-[Bis(2-chloroethyl)amino]-4-phenyl-2H-1,3,2-oxazaphosphorinane 2-oxide, an analogue of Formula II lacking the p-nitro group on the phenyl ring, has been synthesized starting from benzaldehyde or benzoylacetate (Shih, et al. (1978) Hetercycles 9:1277-1285; Boyd, et al. (1980) J. Med. Chem. 23:372-375). Under similar reaction conditions, the nitro-substituted benzaldehyde with malonic acid failed to give the corresponding β-aminocarboxylic acid yielding a complicated reaction product mixture. One product isolated was 4-nitrocinnamic acid, which is the elimination product formed during condensation. To synthesize 4- or 6-(p-nitrophenyl)cyclophosphamides of Formula II, an alternate approach was taken. One advantage of this synthesis was that it provided access to the corresponding dioxa and diaza compounds. In general, the nitroaryl-substituted cyclophosphamide 13 may be prepared according to the following steps of Scheme 3: i) performing a Grignard reaction; ii to v) performing a hydroboration and converting of one or both of the hydroxyl groups to amino; and vi) condensing the 1,3-diols, 3-amino alcohol, or 1,3-diamine with bis(2-chloroethyl)phosphoramidic dichloride.

Preferred embodiments of producing a compound 13 of Formula II include the following. The Grignard reaction may be performed with vinyl magnesium bromide or chloride. Protection of the hydroxyl group in 11 with methoxymethyl or another suitable group is desirable when X is O and Y is NH. Conversion of hydroxyl groups to amino groups may be accomplished in different ways including 1) activation by mesylate followed by an S_{N}2 displacement reaction and 2) by a Mitsunobu reaction using triphenyl phosphine, DEAD, and an azido source (e.g., HN₃ or diphenyl phosphoryl azide). Conversion of azides to amino may be accomplished using reagents like propanedithiol or triphenyl phosphine. Final condensation with bis(2-chloroethyl)phosphoramidic dichloride may be carried out in the presence of a base such as triethylamine.

Representative compounds of Formula II which may be produced in accordance with Scheme 3 include 2-[bis(2-chloroethyl)amino]-4-(p-nitrophenyl)-2H-1,3,2-dioxaphosphorinane 2-oxide (13a); 2-[bis(2-chloroethyl)amino]-6-(p-nitrophenyl)-2H-1,3,2-oxazaphosphorinane 2-oxide (13b); 2-[bis(2-chloroethyl)amino]-4-(p-nitrophenyl)-2H-1,3,2-oxazaphosphorinane 2-oxide (13c); and 2-[bis(2-chloroethyl)amino]-4-(p-nitrophenyl)-2H-1,3,2-diazaphosphorinane 2-oxide (13d).

Monosubstitution at the C-4/C-6 position of cyclophosphamide generated a second chiral center with phosphorus atom being the first chiral center in the ring system. The resultant diastereomeric racemates were referred to as the cis- and trans- (cis=RS/SR; trans=RR/SS) and were assigned from their chromatographic behavior, amide ¹H and ³¹NMR chemical shifts. This was confirmed by X-ray crystallographic analysis using well-known methods (Stec (1982) J. Organophosphorous Chem. 13:145-174; Boyd, et al. (1980) J. Med. Chem. 23 (4): 372-5). Due to the equatorial preference of the phenyl group, compounds 13a-d existed primarily in one solution conformer and were separated through flash silica gel chromatography. The structure of each diastereomer was confirmed by ³¹p NMR.

A further embodiment of the present invention is a method of producing a nitroaryl-substituted phosphoramide compound of Formula II as depicted in Scheme 4.

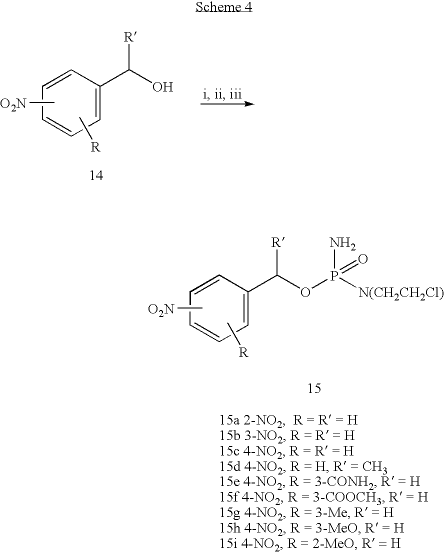

In general, the nitroaryl-substituted phosphoramide 15 may be prepared according to the following steps of Scheme 4: i to iii) condensing a precursor alcohol or amine 14 with bis(2-chloroethyl)phosphoramidic dichloride in the presence of a base and performing hydrolysis or aminolysis with ammonia, H₂NCH₂CH₂Cl or HN(CH₂CH₂Cl)₂. As 14 is an alcohol, a strong base such as butyl lithium is preferably used.

Representative compounds of Formula II which may be produced in accordance with Scheme 4 include 2-nitrobenzyl N,N-bis(2-chloroethyl)phosphordiamidate (15a); 3-nitrobenzyl N,N-bis(2-chloroethyl)phosphordiamidate (15b); 4-nitrobenzyl N,N-bis(2-chloroethyl)phosphordiamidate (15c); 1-(4-nitrophenyl)ethyl N,N-bis(2-chloroethyl)phosphordiamidate (15d); 3-carboxamide-4-nitrobenzyl N,N-bis(2-chloroethyl)phosphordiamidate (15e); 3-methoxycarbonyl-4-nitrobenzyl N,N-bis(2-chloroethyl) phosphordiamidate (15f); 3-methyl-4-nitrobenzyl N,N-bis(2-chloroethyl)phosphordiamidate (15g); 3-methoxy-4-nitrobenzyl N,N-bis(2-chloroethyl)phosphordiamidate (15h); and 2-methoxy-4-nitrobenzyl N,N-bis(2-chloroethyl)phosphordiamidate (15i).

To assess the stability of representative nitrophenyl-substituted phosphoramide compounds of Formulae I and II, each compound was incubated in pH 7.4 phosphate buffer at 37° C. No significant changes were observed in the compounds, with the exception of 13a, as assessed by HPLC analysis over a period of 4 days (<10%), indicating that the compounds, with the exception of 13a, are very stable under physiological conditions.

Upon activation by a reducing agent, the nitroaryl-substituted phosphoramide compounds of Formulae I and II become or release a highly cytotoxic species such as a phosphoramide mustard or like compound. For example, a nitroaryl-substituted phosphoramide compound of Formula I has the cyclophosphamide ring fused with a benzene ring, where the nitro group serves as a strong electron-withdrawing group and is converted to an electron-donating amino or hydroxyamino group upon reduction (Scheme 5). After reduction by a reducing agent, the resulting hydroxyamine or amine 16 relays electrons to the para-position and facilitates the cleavage of the benzylic C—O/NH bond, producing a cytotoxic intermediate (17). The intermediate 17 resembles the phosphoramide mustard (5) produced in the activation process of cyclophosphamide 1 and thereby may function as a cytotoxic alkylating agent. In addition, 17 also possesses additional electrophilic centers that may form cross-links with functionally important macromolecules, providing an additional mechanism for cytotoxicity.

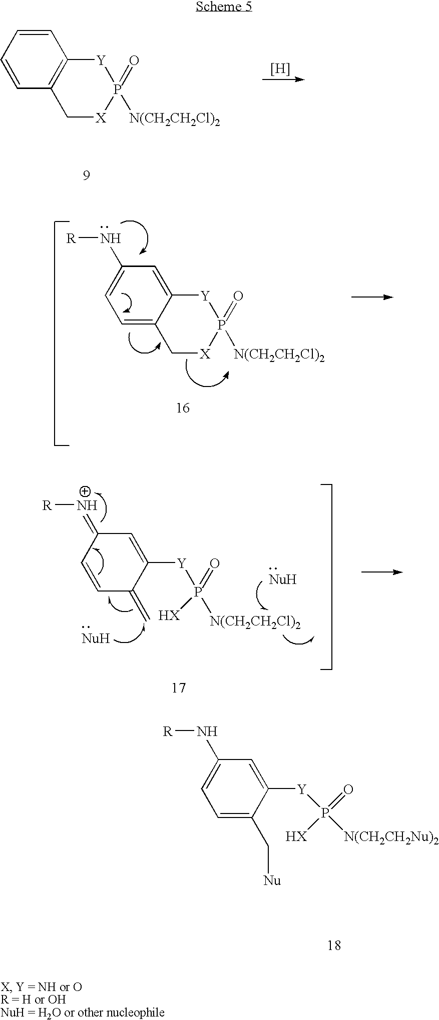

To further analyze the selective reduction of nitroaryl-substituted phosphoramide compounds of the invention, catalytic hydrogenation or NaBH₄ was used in the presence of 10% Pd/C in methanol to selectively reduce the nitro group. Subsequently, the reduced product was characterized with NMR and high resolution MS according to well-established methods (Hu, et al. (2000) Bioorg. Med. Chem. Lett. 10:797-800). In the case of compounds 9a and 9c, where the benzylic carbon is attached to an ester oxygen, reduction gave a complex product mixture, indicating that the corresponding, reduced products were not stable and may undergo the cleavage reactions shown in Scheme 5. However, when the benzylic carbon is attached to a phosphoramide nitrogen (i.e., 9b and 9d), the corresponding, reduced aminobenzocyclophosphamides 19b and 19d were isolated in 97% and 52% yield, respectively (Scheme 6). In addition, both 19b and 19d were found to be similarly stable as compared to their precursors under the same stability testing conditions provided herein.

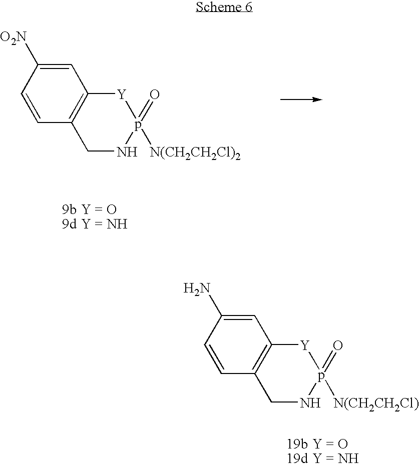

To assess the extent to which the nitroaryl-substituted phosphoramide compounds of the present invention undergo enzymatic reduction, representative compounds of Formulae I and II were incubated with E. coli nitroreductase. Half-lives of each compound were calculated based on the disappearance of the substrate (Table 1) and compared to the half-life of CB1954 (5-(aziridin-1-yl)-2,4-dinitrobenzamide), a substrate for bacterial nitroreductase (Chung-Faye, et al. (2000) Annals of Oncology 11 (Suppl. 4): 133).

TABLE 1

| | NR assay | IC₅₀ (μM)^{b} | | | Ratio^{c} |
|---|---|---|---|---|---|
| Compound | t_{1/2} (min)^{a} | F179 | hDT7 | T116 | (F179/T116) |

Formula I

| | | | | | |
|---|---|---|---|---|---|
| 9a | 24 | >100 | >100 | 2.7 | >36 |
| 9b | 11 | 61 | 48 | 48 | 1.3 |
| 9c | 13 | >100 | >100 | 3.0 | >33 |
| 9d | 7.8^{d} | >100 | >100 | >100 | ˜1 |
| CB1954 | 5.0 | 254 | 1.7 | 0.036 | >2,777 |
| cis-13b | 11.9 | >100 | >100 | 45.3 | >2.2 |
| trans-13b | 2.9 | >100 | >100 | 51.5 | >1.9 |

Formula II

| | | | | | |
|---|---|---|---|---|---|
| cis-13c | 5.2 | 852 | >100 | 0.031 | 27,484 |
| trans-13c | 3.9 | 608 | >100 | 0.027 | 22,519 |
| cis-13d | 6.4 | 56.8 | 46.8 | 4.6 | 12.3 |
| trans-13d | 4.2 | >100 | >100 | 48.3 | >2.1 |
| 15c^{e} | ND^{e} | 62.5 | 27 | 0.003 | 20,833 |
| CB1954 | 5.0 | 254 | 1.7 | 0.036 | 7,056 |

^{a}Half-lives of reduction by E. coli nitroreductase were determined using 0.2 mM of substrate in 10 mM phosphate buffer (pH 7.0) in the presence of 1 mM of NADH at 37° C. in a total volume of 250 μL. The reaction was initiated by the addition of 1.8 μg of E. coli nitroreductase. Aliquots were withdrawn and analyzed by HPLC.
^{b}IC₅₀ values are the concentration required to reduce cell number to 50% of control after 72 hours with drug exposure.
^{c}Ratio of IC₅₀ values (F179/T116) as an indication of activation by E. coli nitroreductase.
^{d}The catalysis seemed to reach an end point of 58%.
^{e}Not determined using the HPLC assay, but the initial velocity was determined using the spectrophotometric assay (see Table 3).

The nitroaryl-substituted phosphoramide compounds of Formula I were found to be substrates of E. coli nitroreductase with half-lives between 7 and 24 minutes, slightly longer than CB1954, which has a half-life of 5 minutes under the same assay conditions. Compound 9d only reached an end point of 58% while all other compounds reached end points of less than 10%. The behavior of compound 9d may indicate that one enantiomer is a better substrate for E. coli nitroreductase than the other. Alternatively, the nitroreductase enzyme may have been inhibited by the reduced product.

Conversely, representative compounds of Formula II were found to be better substrates of E. coli nitroreductase than compounds of Formula I with half-lives predominantly between 2.9 and 6.4, comparable to CB1954.

Representative compounds of Formulae I and II were assayed for cytotoxicity against cells expressing either E. coli nitroreductase (T116) or human quinone oxidoreductase NQ01 (hDT7). Cells were Chinese hamster V79 cells transfected with a bicistronic vector encoding for the E. coli nitroreductase or the human quinone oxidoreductase protein and puromycin resistance protein as the selective marker. F179 cells were transfected with vector only and were used as the controls. The cells were exposed for 72 hours to each test compound (9a-d, 13b-d) and the maximum concentration used was 100 μM.

Compounds of Formula I, with the exception of compounds 9b, which had an IC₅₀ of 61 μM in the control cells, were not cytotoxic at 100 μM in the control cells. The IC₅₀ and the ratios of IC₅₀ (F179/T116) of the test compounds are provided in Table 1. In calculating the ratio of IC₅₀, the value of 100 μM was used for those compounds with an undetermined IC₅₀>100 μM so the ratio was an underestimate. Compounds 9a, 9c, and 9d were not very cytotoxic and were not activated by endogenous mammalian enzymes, at least not those found in V79 cells. Generally, the T116 cells were more cytotoxically affected by the test compounds than the control cells. All compounds, except 9d, tested showed ratios >1 indicating activation by E. coli nitroreductase. Compounds 9b and 9d were found to have similar IC₅₀ values in cells expressing or not expressing E. coli nitroreductase even though both were reduced by E. coli nitroreductase as shown in the enzyme assays. Both of these compounds contain a benzylic nitrogen, instead of a benzylic oxygen, para to the nitro group. Chemical reduction of 9b and 9d produced stable amine products that were not expected to be alkylating agents. Conversely, 9a and 9c with benzylic oxygen at the para position to nitro group gave no clearly identifiable products upon chemical reduction. 9a and 9c were found to be over 30-fold more toxic in E. coli nitroreductase-expressing cells. These results indicate that E. coli nitroreductase-reduction was an important first step but not sufficient for enhanced cytotoxicity in E. coli nitroreductase-expressing cells. Not to be bound by any one theory, it is believed that nitroreductase converts 9a and 9c to their corresponding amino or hydroxylamine analogue, which would then follow the electron “push and pull” mechanism shown in Scheme 5 to produce the observed cytotoxicity. Further, the 33- to 36-fold activation shown by 9a and 9c in E. coli nitroreductase-expressing cells is about 100-fold less than that shown by CB1954.

Compounds of Formula II were shown to have ratios greater >1, indicating activation by E. coli nitroreductase. Compound 13c isomers had low IC₅₀ values similar to CB1954 in E. coli nitroreductase-expressing T116 cells. However, the IC₅₀ values of the 13c isomers in cells not expressing E. coli nitroreductase were about 3-4 times higher than that of CB1954. Compound 15c, another representative compound of Formula II, had an IC₅₀ of 3 nM in E. coli nitroreductase-expressing T116 cells, which was about 10-times more active than CB1954. Overall, compounds 13c (both diastereomeric mixtures) and 15c were over 20,000-fold more selective in targeting E. coli nitroreductase-expressing T116 cells as compared to cells that do not express the enzyme. This level of selectivity was about 3-4 times better than CB1954.

Under similar assay conditions, E. coli nitroreductase-expressing T116 cells were exposed to representative compounds of Formulae I and II for a reduced amount of time, 1 hour. As shown in Table 2, both cis- and trans-13c were shown to have similar activity as that of the control CB1954, while the representative compound 15c was shown to be much more quickly activated with an IC₅₀ as low as 10 nM. This level of activity was about 30-fold better than the control CB1954.

TABLE 2

| | |
|---|---|
| IC₅₀ (μM)^{a} | Ratio^{b} |

| Compound | F179 | T116 | (F179/T116) |
|---|---|---|---|
| cis-13c | >100 | 0.343 | >291 |
| trans-13c | >100 | 0.166 | >602 |
| 15c | >100 | 0.01 | >10,000 |
| CB1954 | >100 | 0.306 | >327 |

^{a}IC₅₀ values are the concentration required to reduce cell number to 50% of control after the cells were exposed to the drug for 1 hour.
^{b}Ratio of IC₅₀ values (F179/T116) as an indication of activation by E. coli nitroreductase.

Representative compounds provided herein were also assayed in human ovarian carcinoma cells (SKOV3) infected with adenovirus expressing E. coli nitroreductase. Cells were infected using multiplicities of infection of 100 pfu/cell relative to uninfected SKOV3 cells and compounds were applied at a maximum concentration of 1 mM. While CB1954 showed a 150-fold selective toxicity in infected versus uninfected SKOV3 cells, a majority of the representative compounds tested (13c, 15c, 15d, 15f-i) showed similar or several fold better selectivity in human ovarian cancer cell lines expressing E. coli nitroreductase than CB1954 (Table 3). These data also indicate that the nitro group is most effective in the para position to the benzylic carbon.

Also shown in Table 3 is the nitroreductase substrate activity of the representative compounds using a spectrophotometric assay. The initial velocity (nmoles/min) was determined by measuring UV absorption change at 340 nm using 200 μM of each compound in the presence of 1 mM NADH and 1.8 μg of E. coli nitroreductase in 10 mM phosphate buffer at pH 7.0 and 37° C. Compound 15i was found to have the best enzyme substrate activity under this condition, followed by 15h, 15c, 15d-A, and 13c. The least active compounds were 15a, 15e and 15f, all with a substituent ortho to the nitro group.

TABLE 3

| | NR Assay^{a} | IC₅₀ (μM) | | |
|---|---|---|---|---|
| | vᵢ | in SKOV3^{b} | | Ratio^{c} |
| Compound | (nmoles/min) | NR− | NR+ | (NR−/NR+) |
| 9b | ND^{d} | 510 | 410 | 1.2 |
| 9c | 1.63 | 540 | 55 | 9.8 |
| cis-13b | ND | >1000 | 510 | >2 |
| trans-13b | ND | 820 | 250 | 3.3 |
| cis-13c | 2.33 | 680 | 4.5 | 151 |
| trans-13c | 3.54 | >1000 | 5 | >200 |
| 15a | 0.46 | 950 | 90 | 11 |
| 15b | 1.23 | 820 | 240 | 3.4 |
| 15c | 2.31 | >1000 | 1.1 | >909 |
| 15d-A | 2.53 | >1000 | 1.8 | >556 |
| 15d-B | 1.65 | >1000 | 4 | >250 |
| 15e | 0.38 | >1000 | 41 | >24 |
| 15f | 0.30 | 400 | 2.1 | 190 |
| 15g | 1.61 | 510 | 3.3 | 155 |
| 15h | 4.40 | 910 | 3.1 | 294 |
| 15i | 12.56 | >1000 | 1.8 | >556 |
| CB1954 | —^{e} | 600 | 4 | 150 |
| ^{a}Nitroreductase substrate activity expressed as the initial velocity (nmoles/min) in the presence of 1.8 □g of E. coli nitroreductase in a total volume of 250 □L by following the UV absorption changes at 340 nm. The results were | | | | |
| #background corrected using the same solution in the absence of the substrate. | | | | |
| ^{b}IC₅₀ values are the concentration required to reduce SKOV3 human ovarian carcinoma cell number to 50% of control after the cells were exposed to the drug for 18 hour. NR− are SKOV3 human ovarian carcinoma cells that were not infected | | | | |
| #with adenovirus expressing E. coli nitroreductase and NR+ are SKOV3 human ovarian carcinoma cells that were infected with adenovirus expressing E. coli nitroreductase using multiplicities of infection of 100 pfu/cell. | | | | |

^{c}Ratio of IC₅₀ values (NR−/NR+) as an indication of activation by E. coli nitroreductase.
^{d}was not determined.
^{e}Could not be determined using the spectrophotometric assay due to the strong UV absorption of CB1954 itself at 340 nm.

The compounds of the Formulae I and II, upon activation by a reducing agent, are cytotoxic to cells and are therefore useful for inhibiting undesirable cell growth. Accordingly, another aspect of the present invention is a pharmaceutical composition containing a nitroaryl-substituted phosphoramide compound of Formula I or II, or a salt thereof, and a pharmaceutically acceptable carrier. Preferably the pharmaceutical composition or pharmaceutical preparation contains an efficacious dose of at least one compound of Formula I or Formula II, or a salt thereof and a pharmaceutically acceptable carrier. Further, the pharmaceutical composition may contain a mixture of compounds of Formulae I and II, or salts thereof, and a pharmaceutically acceptable carrier. The pharmaceutical composition may be administered orally, for example in the form of pills, tablets, lacquered tablets, coated tablets, granules, hard and soft gelatin capsules, solutions, syrups, emulsions, suspensions or aerosol mixtures. Administration may also be carried out rectally (e.g., in the form of a suppository); parenterally (e.g., intravenously, intramuscularly, subcutaneously in the form of injection solutions or infusion solutions, microcapsules, implants or rods); or percutaneously or topically (e.g., in the form of ointments, solutions, emulsions or tinctures, aerosols, or nasal sprays).

The selected pharmaceutically acceptable carrier may be dependent on the route of administration and may be an inert inorganic and/or organic carrier substance and/or additive. For the production of pills, tablets, coated tablets and hard gelatin capsules, the pharmaceutically acceptable carrier may include lactose, corn starch or derivatives thereof, talc, stearic acid or its salts, and the like. Pharmaceutically acceptable carriers for soft gelatin capsules and suppositories include, for example, fats, waxes, semisolid and liquid polyols, natural or hardened oils, and the like. Suitable carriers for the production of solutions, emulsions, or syrups include, but are not limited to, water, alcohols, glycerol, polyols, sucrose, glucose, and vegetable oils. Suitable carriers for microcapsules, implants or rods include copolymers of glycolic acid and lactic acid.

The pharmaceutical compositions, in general, contain about 0.5 to 90% by weight of a compound of Formulae I or II, or a salt thereof. The amount of active ingredient of Formulae I or II, or a salt thereof, in the pharmaceutical composition normally is from about 0.2 mg to about 1000 mg, preferably from about 1 mg to about 500 mg.

In addition to a nitroaryl-substituted phosphoramide compound of Formula I or II, or a salt thereof, and a pharmaceutically acceptable carrier, the pharmaceutical composition may contain an additive or auxiliary substance. Exemplary additives include, for example, fillers, disintegrants, binders, lubricants, wetting agents, stabilizers, emulsifiers, preservatives, sweeteners, colorants, flavorings, aromatizers, thickeners, diluents, buffer substances, solvents, solubilizers, agents for achieving a depot effect, salts for altering the osmotic pressure, coating agents or antioxidants. A generally recognized compendium of methods and ingredients of pharmaceutical compositions is Remington: The Science and Practice of Pharmacy, Alfonso R. Gennaro, editor, 20th ed. Lippincott Williams & Wilkins: Philadelphia, Pa., 2000. Furthermore, one or more other pharmaceutically active agent (e.g., doxorubicin, BCNU, methotrexate, or 5-FU) may be formulated in the pharmaceutical composition of the invention to enhance the desired effect of inhibiting, reducing, or stabilizing undesirable cell growth or proliferation.

The pharmaceutical compositions of the present invention are particularly useful in inhibiting undesirable cell growth or proliferation e.g., inappropriate cell growth resulting in an undesirable benign condition or tumor growth (e.g., benign or malignant). For example, a benign condition is one which results from inappropriate cell growth or angiogenesis including, but not limited to, autoimmune disease, arthritis, graft rejection, inflammatory bowel disease, proliferation induced after medical procedures (e.g., surgery, angioplasty, and the like), diabetic retinopathy, retrolental fibrioplasia, neovascular glaucoma, psoriasis, angiofibromas, hemangiomas, Karposi's sarcoma, and other conditions or dysfunctions characterized by dysregulated endothelial cell division. It is further contemplated that that inhibiting undesirable cell growth may be applied to a benign condition such as obesity to eliminate or reduce undesirable adipose tissue. For example, a composition of the present invention may be targeted to an adipocyte using a gene-directed enzyme prodrug therapy wherein the adipocyte-specific promoter, aP2, drives expression of nitroreductase (Felmer, et al. (2002) J. Endocrinol. 175 (2): 487-98).

Wherein inhibiting undesirable cell growth or proliferation applies to tumor growth, it is intended to include the prevention of the growth of a tumor in a subject or a reduction in the growth of a pre-existing tumor in a subject. The inhibition also may be the inhibition of the metastasis of a tumor from one site to another. In particular, a tumor is intended to encompass both in vitro and in vivo tumors that form in any organ or body part of the subject. The tumors preferably are tumors sensitive to the nitroaryl-substituted phosphoramide compounds of the present invention. Examples of the types of tumors intended to be encompassed by the present invention include, but are not limited to, tumors associated with pancreatic cancer, endometrial cancer, small cell and non-small cell cancer of the lung (including squamous, adneocarcinoma and large cell types), squamous cell cancer of the head and neck, bladder, ovarian and cervical cancers, myeloid and lymphocyltic leukemia, lymphoma, hepatic tumors, medullary thyroid carcinoma, multiple myeloma, melanoma, retinoblastoma, and sarcomas of the soft tissue and bone. The nitroaryl-substituted phosphoramide compounds of the invention are particularly useful for directly treating cancers of the gastrointestinal tract as E. coli bacteria is abundant in these areas and produces a nitroreductase for activation of said compounds.

Accordingly, another aspect of the invention is a method of inhibiting undesirable cell growth or proliferation by administering an effective amount of pharmaceutical composition containing a nitroaryl-substituted phosphoramide compound of Formula I or II, or a salt thereof and a pharmaceutically acceptable carrier. An effective amount of a nitroaryl-substituted phosphoramide-containing composition is considered an amount which inhibits, reduces, or stabilizes the growth or proliferation of undesirable cells and may be determined by measuring rates of cell growth or proliferation, tumor size, or benign tissue mass before and after exposure to said composition.

While hypoxic cells in tumors provide a reducing environment in which the nitroaryl-substituted phosphoramide compounds of the present invention are reduced to deliver a toxic phosphoramide mustard or cytotoxic intermediate, it is contemplated that reducing agents may be provided to the targeted undesirable cell exogenously with the compositions provided herein. Accordingly, in a preferred embodiment of the present invention, a nitroaryl-substituted phosphoramide-containing composition is administered with a reducing agent wherein the nitroaryl-substituted phosphoramide is a prodrug which is directly or indirectly acted upon by the reducing agent to generate a toxic phosphoramide mustard or cytotoxic intermediate.

A reducing agent which directly acts upon a prodrug compound of the invention is typically an enzyme such as nitroreductase, however, any reducing agent which directly acts upon a nitroaryl-substituted phosphoramide-containing prodrug to generate a toxic phosphoramide mustard or cytotoxic intermediate is suitable to carry out the method of the invention. The use of bacterial and human nitroreductases as reducing agents for directly activating a prodrug is well-known in the art (see, e.g., Bilsland, et al. (2003) Oncogene 22 (3): 370-80; Skelly, et al. (2001) Mini Rev. Med. Chem. 1 (3): 293-306).

A reducing agent which indirectly acts upon a nitroaryl-substituted phosphoramide-containing prodrug is one which, for example, promotes hypoxia in a tumor by reducing tumor blood flow. Exemplary reducing agents which indirectly act upon the compounds of the present invention include, but are not limited to, flavone-8-acetic acid (FAA); xanthenone-4-acetic acid (XAA); and 5,6-dimethylxanthenone-4-acetic acid (DMXAA).

The reducing agent may be administered alone or with a targeting agent to direct the reducing agent specifically to the undesirable cells. Such targeting agents may include, for example, antibodies or immunologically reactive fragments thereof, including single-chain antibodies, which are immunospecific for antigens associated with the undesirable cells or for antigens which appear on the organs in which the undesirable cells reside, such as prostate-specific antigen in the case of prostate cancer. In addition, the targeting agents may include ligands for receptors that characterize the undesirable cells such as folic acid for folate receptors in ovarian cancer. Coupling to such targeting agents is conventional and involves standard linking technologies, optionally utilizing commercially available linkers. Any suitable prodrug targeting approach may be employed including antibody- macromolecule-, or gene-directed enzyme prodrug therapy (ADEPT, MDEPT or GDEPT) and may be dependent on the undesirable cell type being targeted.

When using an enzyme to activate the nitroaryl-substituted phosphoramide prodrug, the enzyme may be supplied as a protein or may be generated intracellularly or in situ by supplying an expression system for the enzyme. If the enzyme is administered, methods for administering such proteins are generally known in the art. For example, methods to administer methioninase in particular, in the context of chemotherapy are set forth in U.S. Pat. No. 5,690,929, the contents of which is incorporated herein by reference. Proteins, in general, may be administered by injection, typically intravenous injection or by transmembrane administration, for example, intranasally or using suppositories. Other modes of administration are also possible, including oral administration provided adequate protection from hydrolysis is included in the formulation. Such methods are generally known in the art as described in Remington: The Science and Practice of Pharmacy, Alfonso R. Gennaro, editor, 20th ed. Lippincott Williams & Wilkins: Philadelphia, Pa., 2000.

When an enzyme is to be generated intracellularly or in situ, a suitable nucleic acid molecule containing the nucleotide sequence encoding the enzyme is administered. Suitable modes of administration include injection, topical administration in formulations that include agents which enhance transmembrane or transdermal transit or any other appropriate and convenient method consistent with the undesirable cells being treated and the nature of the formulation, as will be understood by the ordinary practitioner.

The nucleic acid molecule for delivery of the nucleic acid sequence encoding the reducing enzyme is typically a vector, most commonly a viral vector, although naked DNA can, in some instances, be used. The viral vectors may be retroviral vectors, which preferentially replicate in rapidly proliferating cells, thus conferring specificity for tumor cells on the vector, or may include adenoviral vectors or other conventional vector-based molecules. Specificity in this case may be conferred by localized administration and/or by placing the expression of the nucleotide sequence encoding the enzyme under control of a promoter which is operable selectively in the undesirable cells (e.g., adipocyte-specific promoter, aP2).

Suitable viral vector constructs are known in the art. For example, vectors derived from a parvovirus (U.S. Pat. Nos. 5,252,479 and 5,624,820), a paramyxovirus such as simian virus 5 (SV5) (U.S. Pat. No. 5,962,274), a retrovirus such as HIV (U.S. Pat. Nos. 5,753,499 and 5,888,767), and a baculovirus such as a nuclear polyhedrosis virus (U.S. Pat. No. 5,674,747) may be used. Vectors derived from adenovirus (U.S. Pat. Nos. 5,670,488, 5,817,492, 5,820,868, 5,856,152 and 5,981,225) are also contemplated herein.

The nucleic acid molecule may be delivered directly to a tissue of the host animal by injection, by gene gun technology or by lipid mediated delivery technology. The injection can be conducted via a needle or other injection devices. The gene gun technology is disclosed in U.S. Pat. No. 5,302,509 and the lipid mediated delivery technology is disclosed in U.S. Pat. No. 5,703,055.

While the nitroaryl-substituted phosphoramide prodrug and the reducing agent may be delivered concomitantly, it is preferred that the reducing agent be provided first, followed by administration of the nitroaryl-substituted phosphoramide prodrug to precondition the undesirable cells to generate the toxic phosphoramide mustard or intermediate.

Those of ordinary skill in the art may readily optimize effective doses and co-administration regimens as determined by good medical practice and the clinical condition of the individual patient. Regardless of the manner of administration, it may be appreciated that the actual preferred amounts of active compound in a specific case will vary according to the efficacy of the specific compound employed, the particular compositions formulated, the route of administration. The specific dose for a particular patient depends on age, body weight, general state of health, on diet, on the timing and route of administration, on the rate of excretion, and on medicaments used in combination and the severity of the particular disorder to which the therapy is applied. Dosages for a given subject may be determined using conventional considerations, e.g., by customary comparison of the differential activities of the subject compounds and of a known agent, such as by means of an appropriate conventional pharmacological protocol.

### EXAMPLE 1

### General Methods

Air-sensitive materials were transferred by syringe or cannula under an argon atmosphere. Except for redistillation prior to use, solvents were either ACS reagent grade or HPLC grade. Tetrahydrofuran (THF) was dried over sodium/benzophenone. Triethylamine, dichloromethane and ethyl acetate were dried over calcium hydride. Pyridine was dried over potassium hydroxide and distilled over calcium hydride. N,N-dimethylformamide (DMF) was dried over a 4 Å molecular sieve at least for one week prior to use. Unless otherwise indicated, all reactions were magnetically stirred and monitored by thin-layer chromatography (TLC) using 0.25 mm Whatman precoated silica gel plates. TLC plates were visualized using either 7% (w/w) ethanolic phosphomolybdic acid or 1% (w/w) aqueous potassium permagnate containing 1% (w/w) NaHCO₃. Flash column chromatography was performed using silica gel (Merck 230-400 mesh). Yields refer to chromatographically and spectroscopically (¹H NMR) homogeneous materials, unless otherwise indicated. All reagents were purchased at the highest commercial quality and used without further purification.

Infrared spectra were recorded with a Perkin-Elmer model 1600 series FTIR spectrometer using polystyrene as an external standard. Infrared absorbance was reported in reciprocal centimeters (cm⁻¹). All ¹H and ¹³C, and ³¹p NMR spectra were recorded on a Varian Gemini 300 MHz spectrometer at ambient temperature and calibrated using residual undeuterated solvents as the internal reference. Chemical shifts (300 MHz for ¹H and 75 MHz for ¹³C) are reported in parts per million (δ) relative to CDCl₃ (δ 7.27 for ¹H and 77.2 for ¹³C) and CD₃OD (δ 3.3 for ¹H and 49.0 for ¹³C). Coupling constants (J values) are given in hertz (Hz). The following abbreviations were used to explain the multiplicities: s=singlet; d=doublet; t=triplet; q quartet; p=quintet; m=multiplet; br=broad. Mass spectral data were obtained from the University of Kansas Mass Spectrometry Laboratory (Lawrence, Kans.).

### EXAMPLE 2

### Synthesis of 7-nitro-2-[bis(2-chloroethyl) amino]-1,3,2-benzodioxaphosphorinane-2-oxide (9a)

The dioxa analogue 9a was synthesized starting from 2-methyl-5-nitrophenol. Acetylation with acetic anhydride followed by bromination with N-bromosuccinimide afforded 2-acetoxy-4-nitrobenzyl bromide in 76% yield for the two steps. Complete hydrolysis of both the ester and the bromide in the acetic acid, 2-bromomethyl-5-nitrophenyl ester using CaCO₃ in H₂O-dioxane (1:1) gave 2-hydroxy-4-nitrobenzyl alcohol in 82% yield. Subsequent triethylamine-mediated cyclization with bis(2-chloroethyl)phosphoramidic dichloride gave the desired 7-nitro-2-[bis(2-chloroethyl)amino]-1,3,2-benzodioxaphosphorinane-2-oxide (9a) in 55% yield. The overall yield for the synthesis of 9a before optimization is 34%.

Acetic acid, 2-bromomethyl-5-nitrophenyl ester. 2-Methyl-5-nitrophenol (2.5 g, 13 mmol) was dissolved in 50 mL of acetic anhydride (10 eq) and immersed in an ice water bath. After the addition of pyridine (2 mL, 1.2 eq), the reaction mixture was stirred at room temperature for 6 hours. Excess acetic anhydride was removed under reduced pressure and the residue was dissolved in 100 mL of CH₂Cl₂, washed with saturated NaHCO₃, water, dried over Na₂SO₄. 2-Methyl-5-nitrophenyl acetate was obtained as a white solid (2.9 g, 91%). m.p. 68-72° C., ¹H NMR (300 MHz, CDCl₃) δ 8.04 (d, 1H, J=8.4 Hz), 7.93 (s, 1H), 7.40 (d, 1H, J=8.4 Hz), 2.37 (s, 3H), 2.29 (s, 3H); MS (FAB⁺) m/z (relative intensity) 196 (MH⁺, 12.9), 195 (50.8), 152 (54.1), 135 (70.5), 119 (100).

2-Methyl-5-nitrophenyl acetate (2.9 g, 14.9 mmol) and N-bromosuccinimide (2.65 g, 14.9 mmol) were suspended in 50 mL of carbon tetrachloride, and photolyzed with a 300 watt lamp under N₂ for 14 hours. The reaction mixture was then diluted with 50 mL of methylene chloride, washed with water and brine, dried over anhydrous Na₂SO₄. The residue after removal of solvents was purified through flash column chromatography to afford the desired acetic acid, 2-bromomethyl-5-nitrophenyl ester product (3.27 g, 83%). m.p. 76.5-78° C. ¹H NMR (300 MHz, CDCl₃) δ 8.09-8.04 (m, 2H), 7.60 (d, 1H, J=8.4 Hz), 4.44 (s, 2H), 2.43 (s, 3H). MS (FAB⁺) m/z (relative intensity) 196 (MH⁺-Br, 7.9), 195 (82.3).

2-Hydroxy-4-Nitrobenzyl alcohol. Acetic acid, 2-bromomethyl-5-nitrophenyl ester (200 mg, 0.7 mmol) dissolved in 2 mL of dioxane, was mixed with 5.2 equiv of CaCO₃ in 2 mL of H₂O and the reaction mixture was heated to reflux for 3 hours. After the disappearance of starting material as shown by TLC, dioxane was removed by evaporation and the residue was treated with 5 mL of 2 N HCl and extracted with 30 mL of EtOAc. The combined extract was washed with brine (3×30 mL) and dried over anhydrous Na₂SO₄. Final separation through flash column chromatography afforded the desired product 2-hydroxy-4-nitrobenzyl alcohol (101 mg, 81.9%). m.p. 145-149° C. ¹H NMR (300 MHz, CDCl₃), δ 7.70 (s, 1H), 7.69 (d, 1H, J=9.0 Hz), 7.34 (d, 1H, J=9.0 Hz), 4.81 (s, 2H), 4.53 (s, 1H), 2.20 (s, 1H). MS (EI) m/z (relative intensity) 169 (41.6, M+), 151 (100), 105 (54.4), 77 (78.4).

7-Nitro-2-[bis(2-chloroethyl)amino]-1,3,2-benzodioxa-phosphorinane-2-oxide (9a). 2-Hydroxy-4-nitrobenzyl alcohol (100 mg, 0.59 mmol) was dissolved in 1 mL of EtOAc and mixed with 2.0 equiv of Et₃N and a solution of bis(2-chloroethyl)phosphamidic dichloride (153 mg, 1.0 equiv) in 1 mL of EtOAc. The mixture was stirred at room temperature for 18 hours. After removal of the precipitate through filtration, the filtrate was purified by flash column chromatography to give 1,3-dioxa analogue 9a as a yellow oil (114.7 mg, 54.6%). ¹H NMR (300 MHz, CDCl₃) δ 8.02 (d, 1H, J=8.4 Hz), 7.92 (s, 1H). 7.32 (d, 1H, J=8.4 Hz), 5.71-5.24 (m, 2H), 3.67 (t, 4H, J=6.6 Hz), 3.55-3.46 (m, 4H); IR (neat) 2960-2820, 1520, 1420, 1340, 1260, 970, 840 and 726 cm⁻¹; MS (FAB⁺) m/z (relative intensity) 355 (MH⁺, 12.6), 307 (16.2), 289(8.9), 154(100); HRMS (FAB⁺) m/z calc'd for C₁₁H₁₄Cl₂N₂O₅P: 355.0017, found: 354.9992.

### EXAMPLE 3

### Synthesis of 7-nitro-2-[bis(2-chloroethyl)amino]-1,3,2-benzoxazaphos-phorinane-2-oxide (9b)

The benzo[e]cyclophosphamide analogue 9b was synthesized using the Gabriel synthesis of primary amines by converting the bromide of acetic acid, 2-bromomethyl-5-nitrophenyl ester via intermediate 2-acetoxy-4-nitro-α-phthalimido toluene to 2-hydroxy-4-nitrobenzylamine in 32% yield. Subsequent triethylamine-mediated cyclization with bis(2-chloroethyl)phosphoramidic dichloride gave the desired 7-nitro-2-[bis(2-chloroethyl)amino]-1,3,2-benzoxazaphos-phorinane-2-oxide 9b in 62% yield. The overall yield before optimization for the synthesis of 9b is 15%.

2-Acetoxy-4-nitro-α-phthalimido toluene. Acetic acid, 2-bromomethyl-5-nitrophenyl ester (3.9 g, 14.2 mmol) was dissolved in 50 mL of toluene and mixed with potassium phthalimide (2.63 g, 1.2 equiv) and 18-crown-6 (375 mg, 0.1 equiv). The suspension was stirred at room temperature for 20 hours. The reaction mixture was then diluted with 50 mL of water and extracted with methylene dichloride. The CH₂Cl₂ extract was washed with 5% citric acid, saturated NaHCO₃, and H₂O. After drying over anhydrous Na₂SO₄ and removal of solvent, the residue was purified through flash column chromatography to give the desired 2-acetoxy-4-nitro-α-phthalimido toluene product (2.5 g. 52%). m.p. 175-178° C., ¹H NMR (300 MHz, CDCl₃) δ 8.17-7.73 (m, 7H,), 4.89 (s, 2H), 2.47 (s, 3H). MS (FAB⁺) m/z (relative intensive) 341 (MH⁺, 5), 299 (7), 195 (33), 152 (39), 135 (100). HRMS (FAB⁺) m/z calc'd for C₁₇H₁₃N₂O₆: 341.0773, found: 341.0773.

2-Hydroxy-4-nitrobenzylamine. To a solution of compound 2-acetoxy-4-nitro-α-phthalimido toluene (2.5 g, 7.35 mmol) in 50 mL of 1:1 mixture of CH₂Cl₂ and CH₃OH was added 2.4 equiv of hydrazine. The reaction mixture was stirred at room temperature for 14 hours. After removal of solvent under reduced pressure, the residue was treated with 6 N HCl (50 mL) and stirred at room temperature for 1 hour. The filtrate was neutralized to pH=7 with aqueous NaOH solution and extracted with EtOAc. The combined EtOAc extract was dried over anhydrous Na₂SO₄ and concentrated to dryness to afford the desired 2-hydroxy-4-nitrobenzylamine product (0.752 g, 60.6%). m.p. 210-215° C. ¹H NMR (300 MHz, CD₃OD) δ 7.45 (s, 1H), 7.42 (d, 1H, J=8.1 Hz), 7.26 (d, 1H, J=8.1 Hz), 4.00 (s, 2H). ¹H NMR (300 MHz, DMSO) δ 7.55 (d, 1H, J=8.4 Hz), 7.43 (s, 1H), 7.36 (d, 1H, J=8.4 Hz), 3.91 (s, 2H). MS (FAB⁺) m/z (relative intensity) 169 (MH⁺, 7), 154 (100), 136 (69). HRMS (FAB⁺) m/z calc'd for C₇H₉N₂O₃: 169.0613, found: 169.0613.

7-Nitro-2-[bis(2-chloroethyl)amino]-1,3,2-benzoxazaphosphorinane-2-oxide (9b). To a solution of 2-hydroxy-4-nitrobenzylamine (752 mg, 4.47 mmol) and 2.0 equiv of Et₃N in 20 mL of EtOAc was added dropwise with stirring a solution of 1.0 equiv of bis(2-chloroethyl)phosphoramidic dichloride (1.16 g, 4.47 mmol) in 20 mL of EtOAc. After stirring was continued for 14 hours, the precipitate was removed by suction filtration and the filtrate was concentrated under reduced pressure. The residue was purified by flash column chromatography to afford the desired product 9b (974 mg, 61.9%). m.p. 123-126° C. ¹H NMR (300 MHz, CDCl₃) δ 7.97 (d, 1H, J=8.1 Hz), 7.90 (s, 1H), 7.29 (d, 1H, J=8.1 Hz), 4,61-4,31 (m, 2H), 3.80 (s, 1H), 3.72-3.59 (m, 4H), 3.57-3.47 (m, 4H). IR (neat) 3100, 1480, 1304, 1175, 1045, 925 and 804 cm⁻¹; MS (FAB⁺) m/z (relative intensity) 354 (MH⁺, 3.3), 309 (6.5), 195 (28), 152 (68), 135 (90), 119 (100). HRMS (FAB⁺) m/z calc'd for C₁₁H₁₅N₃O₄Cl₂P: 354.0177, found: 354.0181.

### EXAMPLE 4

### Synthesis of 7-nitro-2-[bis(2-chloroethyl)amino]-3,1,2-benzoxazaphosphorinane-2-oxide (9c)

The benzo[e]cyclophosphamide analogue, 7-nitro-2-[bis(2-chloroethyl)amino]-3,1,2-benzoxazaphosphorinane-2-oxide (9c) was synthesized starting from 2-methyl-5-nitroaniline using a similar series of reactions provided for the synthesis of 9a and 9b. The overall yield for the synthesis of 9c before optimization was 4.5%. The overall yields of 9c and 9d syntheses are limited by formation of the phosphorinane ring system. The yields reported in literature for the cyclization and formation of similar systems vary from 15% to around 50% (Ludeman and Zon (1975) J. Med. Chem. 18:1251-1253; Takamizawa and Matsumoto (1978) Chem. Pharm. Bull. 26:790-797; Shih, et al. (1978) Heterocycles 9:1277-1285; Borch and Canute (1991) J. Med. Chem. 34:3044-3052; Viljanen, et al. (1998) J. Org. Chem. 63:168-627)

2-Acetamido-4-nitrobenzyl bromide. To a solution of 2-methyl-5-nitroaniline (3.04 g, 2 mmol) in 50 mL of CHCl₃ were added Ac₂O (10 equiv) and pyridine (1.78 mL, 1.1 equiv). The reaction mixture was stirred at room temperature overnight. After concentration under reduced pressure, the residue was dissolved in 100 mL of CH₂Cl₂, washed with water, saturated NaHCO₃ and water, and dried over anhydrous Na₂SO₄. After removal of solvent, the residue was triturated with CCl₄ to give the desired product 2-acetamido-4-nitrotoluene as a solid (3.36 g, 84%). m.p. 154-155° C. ¹H NMR (300 MHz, CDCl₃) δ 8.76 (s, 1H), 7.94 (d, 1H, J=8.1 Hz), 7.34 (d, 1H, J=8.1 Hz), 7.09 (br, 1H), 2.37 (s, 3H), 2.26 (s, 3H).

2-Acetamido-4-nitrotoluene (1.0 g, 3.66 mmol) and N-bromosuccinimide (0.78 g, 1.2 equiv) were suspended in 100 mL of CCl₄ and photolized with a 300 watt lamp under N₂ for 20 hours. After removal of solvent under reduced pressure, the residue was subjected to flash column chromatography to afford the desired 2-acetamido-4-nitrobenzyl bromide product (0.46 g, 55.6% after recovery of 0.2 g of starting material). m.p. 187.5-189° C. ¹H NMR (300 MHz, CDCl₃) δ 8.84 (s, 1H), 8.00 (d, 1H, J=8.4 Hz), 7.54 (br, 1H), 7.50 (d, 1H, J=8.4 Hz), 4.52 (s, 2H), 2.32 (s, 3H); MS (FAB⁺) m/z (relative intensity) 273 (MH⁺, 5.6), 195 (25.7), 153 (33.1), 135 (100).

2-Amino-4-nitrobenzyl alcohol. 2-Acetamido-4-nitrobenzyl bromide (163 mg, 0.6 mmol) dissolved in 2 mL dioxane was mixed with a suspension of CaCO₃ (358.5 mg, 3.6 mmol) in 2 mL of water. The mixture was then heated up to reflux for 3 hours until all starting material disappeared as monitored by TLC. After removal of solvent under reduced pressure, the residue was treated with 2 mL of 2 N HCl and extracted with CH₂Cl₂. The organic extract was dried over Na₂SO₄ and subjected to flash column chromatography to give 2-acetamido-4-nitrobenzyl alcohol (53.2 mg, 42.2%). ¹H NMR (300 MHz, CDCl₃) δ 9.01 (d, 1H, J=2.1 Hz), 8.87 (br, 1H), 7.91 (dd, 1H, J1=2.1 Hz, J2=8.1 Hz), 7.32 (d, 1H, J=8.1 Hz), 4.82 (d, 2H, J=5.7 Hz), 2.53 (t, 1H, J=5.7 Hz), 2.24 (s, 3H). MS (FAB⁺) m/z (relative intensity) 211 (MH⁺, 7.5), 195 (34.0), 152 (42.0), 135 (100).

2-Acetamido-4-nitrobenzyl alcohol (53.2 mg, 0.316 mmol) was treated with 1 mL of 6 N HCl and the reaction mixture was stirred at room temperature overnight. After neutralization with 6 N aqueous NaOH solution to pH 10, the reaction mixture was extracted with EtOAc, dried over Na₂SO₄, purified through flash column chromatography to give desired 2-amino-4-nitrobenzyl alcohol product (46 mg, 100%). m.p. 178-180° C. ¹H NMR (300 MHz, CDCl₃) δ 7.56-7.51 (m, 2H, aromatic), 7.20 (d, 1H, J=8.1 Hz, aromatic), 4.74 (d, 2H, J=4.5 Hz), 4.52 (br s, 2H), 1.72 (t, 1H, J=4.5 Hz). MS (EI) m/z (relative intensity) 168 (M+, 100), 150 (60.8).

7-Nitro-2-[bis(2-chloroethyl)amino]-3,1,2-benzoxazaphosphorinane-2-oxide (9c). To a solution of 2-amino-4-nitrobenzyl alcohol (46 mg, 0.27 mmol) in 0.5 mL of EtOAc were added with stirring Et₃N (54.6 mg, 0.54 mmol) and bis(2-chloroethyl)phosphoramidic dichloride (70.8 mg, 0.27 mmol) in 0.5 mL EtOAc. After 48 hours, the precipitate was removed by suction filtration and the filtrate was concentrated under reduced pressure. The residue was purified through flash column chromatography to give the desired product 9c as a yellow solid (21.6 mg, 22.5%). m.p. 138-142° C. ¹H NMR (300 MHz, CDCl₃) δ 7.78 (dd, 1H, J1=2.4 Hz, J2=8.1 Hz), 7.69 (d, 1H, J=2.4 Hz), 7.22 (d, 1H, J=8.1 Hz), 6.57 (d, 1H), 5.56-5.07 (m, 2H), 3.69-3.62 (m, 4H), 3.48-3.39 (m, 4H). IR (neat) 3600-3000 (broad), 2930, 2860, 1600, 1520, 1450, 1340, 1220, 970, 880, 820, and 735 cm⁻¹. MS (FAB⁺) m/z (relative intensity) 354 (MH⁺, 4.9), 307 (20.0), 289 (12.6), 154 (100), 136 (98.8). HRMS (FAB⁺) m/z calc'd for C₁₁H₁₅Cl₂N₃O₄P: 354.0177, found: 354.0162.

### EXAMPLE 5

### Synthesis of 7-nitro-2-[bis(2-chloroethyl)amino]-1,3,2-benzodiazaphosphorinane-2-oxide (9d)

The diaza analogue, 7-nitro-2-[bis(2-chloroethyl)amino]-1,3,2-benzodiazaphosphorinane-2-oxide (9d) was synthesized starting from 2-methyl-5-nitroaniline using a similar series of reactions provided for the synthesis of 9a and 9b. The overall yield for the synthesis of 9d before optimization was 6.8%.

2-Acetamido-4-nitro-α-phthalimido toluene. A solution of 2-acetamido-4-nitrobenzyl bromide (45.9 mg, 0.168 mmol) in 2 mL of THF was mixed with 1.5 equiv of potassium phthalimide (146.6 mg) and a catalytic amount of 18-Crown-6 (4.4 mg, 0.1 equiv). The reaction mixture was stirred at room temperature for 24 hours. After removal of solvent, the residue was taken up in 20 mL of CH₂Cl₂, washed with 5% citric acid, saturated NaHCO₃, and water, and dried over Na₂SO₄. Purification through flash column chromatography afforded the desired product 18 (37.2 mg, 73.3% after recovery of 5 mg of starting material). m.p. 221.3-224° C. ¹H NMR (300 MHz, CDCl₃) δ 8.97 (s, 1H), 7.96-7.76 (m, 6H), 4.88 (s, 2H), 2.39 (s, 3H). MS (FAB⁺) m/z (relative intensity) 340 (MH⁺, 6.2), 307 (16.9), 289 (9.9), 273 (4.0), 154 (100), 136 (67.2).

2-Amino-4-nitrobenzylamine. 2-Acetamido-4-nitro-α-phthalimido toluene (50 mg, 0.15 mmol) was suspended in 2 mL of 6 N HCl and stirred at 50° C. for 5 hours. After filtration to remove the solid, the filtrate was neutralized to pH 10 and extracted with EtOAc. The EtOAc extract was dried over anhydrous Na₂SO₄. Removal of EtOAc afforded the desired 2-amino-4-nitrobenzylamine product (15.7 mg, 63.8%). ¹H NMR (300 MHz, CDCl₃) δ 7.51 (dd, 1H, J1=2.4 Hz, J2=8.1 Hz), 7.49 (d, 1H, J=2.4 Hz), 7.15 (d, 1H, J=8.1 Hz), 3.97 (s, 2H).

7-Nitro-2-[bis(2-chloroethyl)amino]-1,3,2-benzodiazaphosphorinane-2-oxide (9d). To a solution of 2-amino-4-nitrobenzylamine (358 mg, 2.14 mmol) in 8 mL of EtOAc were added with stirring Et₃N (433 mg, 4.28 mmol) and bis(2-chloroethyl)-phosphoramidic dichloride (554 mg, 2.14 mmol) in 2 mL of EtOAc. After the reaction mixture was stirred for an additional 3 hours, the precipitate was removed by suction filtration and the filtrate was concentrated under reduced pressure. The residue was purified through flash column chromatography to give the desired product 9d as a yellow solid (263 mg, 34.6%). m.p. 168-169.5° C. ¹H NMR (300 MHz, CDCl₃) δ 7.74 (dd, 1H, J1=2.4 Hz, J2=8.4 Hz), 7.65 (d, 1H, J=2.4 Hz), 7.16 (d, 1H, J=8.4 Hz), 6.23 (br s, 1H), 4.46-4.12 (m, 2H), 3.66 (t, 4H, J=5.7 Hz), 3.48-3.37 (m, 4H), 3.24 (br s, 1H). MS (FAB⁺) m/z (relative intensity) 324 (MH⁺, 4.2), 307 (17.9), 289 (10.4), 273 (4.6), 154 (100), 147 (58.2), 136 (68.7). HRMS (FAB⁺) m/z calc'd for C₁₁H₁₇Cl₂N₃O₂P: 324.0435, found: 324.0435.

### EXAMPLE 6

### Synthesis of 2-[Bis(2-chloroethyl)amino]-4-(p-nitrophenyl)-2H-1,3,2-dioxaphosphorinane 2-oxide (13a)

The synthesis of the dioxa analogue 13a was accomplished in four steps starting from p-nitrobenzaldehyde. Grignard reaction with vinylmagnesium bromide gave 1-(4-Nitrophenyl)-prop-2-en-1-ol in 95% yield. Hydroboration of the 1-(4-Nitrophenyl)-prop-2-en-1-ol with borane followed by basic hydroperoxide oxidation afforded the 1-(4-Nitrophenyl)-propane-1,3-diol in 82% yield. Cyclization of 1-(4-Nitrophenyl)-propane-1,3-diol with bis(2-chloroethyl)phosphoramidic dichloride in the presence of 2 eq of Et₃N gave the crude product 13a, which was separated using flash column chromatography on silica gel with EtOAc-petroleum ether as the eluent to give analytically pure, faster eluting diastereomer cis-13a (R_{f}=0.26 with 1:1 petroleum ether: EtOAc) in 8.9% yield and the slower eluting diastereomer trans-13a (R_{f}=0.20 with 1:1 petroleum ether: EtOAc) in 5.1% yield, with 82.4% starting material recovered. Both diastereomers were an oil and NMR confirmed their structures.

1-(4-Nitrophenyl)-prop-2-en-1-ol. To the solution of p-nitrobenzaldehyde (855 mg, 5.66 mmol) in 20 mL of freshly redistilled THF was added dropwise to vinyl magnesium bromide solution (1 M in THF, 1.2 eq.) under −78° C. The reaction was stirred at −50° C. for 40 minutes and then quenched by saturated ammonium chloride. After the addition of 100 mL of ethyl acetate, the organic phase was washed by brine and dried over anhydrous sodium sulfate. After filtration and removal of the organic solvent under reduced pressure, the crude product was purified through flash silica gel column chromatography (hexane/ethyl acetate, 3/1 to 1/1) to afford desired alcohol (968 mg, 95%). m.p. (EtOAc) 54-55.5° C.; ¹H NMR (300 MHz, CDCl₃) δ 8.15 (d, J=8.1 Hz, 2H), 7.51 (d, J=8.2 Hz, 2H), 6.02-5.90 (m, 1H), 5.40-5.20 (m, 3H), 2.80 (br s, 1H, OH); IR (KBr): 3300 (br), 1580, 1500, 1330, 1250, 1030, 920, 840, 730 cm⁻¹; MS (FAB⁺, NBA) m/z (relative intensity) 180.1 (M+1, 18.9), 162.0 (M-OH, 18.8); HRMS (FAB⁺) m/z calc'd for C₉H₁₀NO₃ (M+1) 180.0661, found 180.0670.

1-(4-Nitrophenyl)-propane-1,3-diol. To the solution of 1-(4-nitrophenyl)-prop-2-en-1-ol (3.1 g, 17.3 mmol) in 150 mL of freshly distilled THF was added slowly a solution of borane in THF (1 M, 1.0 eq.) at 0° C. The reaction was stirred at 0° C. for 20 hours. To the reaction mixture was then added 19 mL of 3 N sodium hydroxide and 19 mL of 30% hydrogen peroxide. After stirring for an additional 30 minutes, ethyl acetate was added and the organic phase was washed with brine and dried over anhydrous sodium sulfate. After filtration and removal of the organic solvent on rotavap, the crude product was purified through flash silica gel column chromatography (hexane/acetate, 2/1 to 1/5) to afford the desired diol (2.8 g, 82%). ¹H NMR (300 MHz, CDCl₃) δ 8.20 (d, J=8 Hz, 2H), 7.55 (d, J=8 Hz, 2H), 5.1 (t, J=7 Hz, 1H) 3.90 (m, 2H), 3.65 (br s, 1H, OH), 2.40 (br s, 1H, OH), 1.96 (m, 2H); IR (KBr): 3400 (br), 1500, 1320 cm⁻¹; MS (FAB⁺, NBA) m/z (relative intensity) 198.1 (M+1, 11.0), 180.1 (M-OH, 13.6); HRMS (FAB⁺) m/z calc'd for C₉H₁₂NO₄ (M+1) 198.0766, found 198.0788.

2-[Bis(2-chloroethyl)amino]-4-(p-nitrophenyl)-2H-1,3,2-dioxaphosphorinane 2-oxide (13a). A solution of 1-(4-nitrophenyl)-propane-1,3-diol (395 mg, 2.0 mmol) in 20 mL of anhydrous ethyl acetate was charged with triethylamine (2 eq., 557 μL) and cooled in ice-water bath for 10 minutes, then was treated with a solution of bis(2-chloroethyl)phosphoramidic dichloride (1 eq., 519 mg) in 10 mL of ethyl acetate. The reaction was stirred at ambient temperature for 48 hours and subsequently partitioned between ethyl acetate and brine. After drying over anhydrous sodium sulfate and filtration, the organic layer was concentrated to afford the crude product. Purification via flash silica gel column chromatography (hexane/ethyl acetate, 6/5 to 5/6) gave two chromatographically separable isomers: the cis-13a (68.3 mg, 8.9%) and the trans-13a (39 mg, 5.1%) upon recovering 326 mg of the starting material.

cis-13a: ¹H NMR (300 MHz, CDCl₃) δ 8.22 (d, J=8.1 Hz, 2H), 7.57 (d, J=8.1 Hz, 2H), 5.05-4.77 (m, 1H), 4.67-4.23 (m, 2H), 3.82-3.47 (m, 8H), 2.23-2.00 (m, 2H); 31p NMR (300 MHz, CDCl₃) δ 11.74 (s); IR (KBr) 1710, 1510 cm⁻¹; IR (KBr) 1710, 1510, 1340 cm⁻¹; MS (FAB⁺, NBA) m/z (relative intensity) 383.0 (M+1, 3.6), 385.0 (M+3, 1.6); HRMS (FAB⁺) m/z calc'd for C₁₃H₁₈N₂O₅PCl₂ (M+1) 383.0330, found 383.0293.

trans-13a: δ 8.20 (d, J=8.0 Hz, 2H), 7.60 (d, J=8.0 Hz, 2H), 5.10-4.75 (m, 1H), 4.60-4.30 (m, 2H), 3.80-3.45 (m, 8H), 2.30-1.95 (m, 2H); 31p NMR (300 MHz, CDCl₃) δ 22.47 (s); IR (KBr) 1690, 1590, 1500, 1430 cm⁻¹; MS (FAB⁺, NBA) m/z (relative intensity) 382.9 (M+1, 2.4), 385.0 (M+3, 0.7); HRMS (FAB⁺) m/z calc'd for C₁₃H₁₈N₂O₅PCl₂ (M+1) 383.0330 found 383.0325.

### EXAMPLE 7

### Synthesis of 2-[Bis(2-chloroethyl)amino]-4-(p-nitrophenyl)-2H-1,3,2-oxazaphosphorinane 2-oxide (13b).

For the synthesis of 4-(p-nitrophenyl) cyclophosphamide (13b), the primary hydroxyl group was first selectively protected as the silyl ether to give 3-(tert-butyldiphenylsilanyloxy)-1-(4-nitrophenyl)-propan-1-ol, where the secondary hydroxyl group was then converted to the azido group using the Mitsunobu reaction condition. Several conditions including (CF₃SO₂)₂O/py-NaN₃, MsCl/NEt₃-NaN₃, PPh₃/DEAD-(PhO)₂PON₃, and DBU-(PhO)₂PON₃ failed to give the desired azide. This difficulty could be attributed to facile elimination of activated ester intermediate. Reduction of the azido group to amino and removal of the silyl protecting group afforded the 3-amino-3-(p-nitrophenyl)-1-propanol. Final cyclization of the 1,3-aminoalcohol with bis(2-chloroethyl)phosphoramidic dichloride gave the desired product 13b. Two diastereomers were separated using silica gel chromatography.

3-(tert-Butyldiphenylsilanyloxy)-1-(4-nitrophenyl)-propan-1-ol. To a solution of 1-(4-nitrophenyl)-propane-1,3-diol (630 mg, 2.55 mmol) in 25 mL of dry DMF was added imidazole (5 eq., 866 mg). After cooling to −40° C., the reaction mixture was treated with tert-butyldiphenylsilyl chloride (1.05 eq., 683 μL), slowly warmed up to −20° C., and stirred for an additional 1.2 hours. The reaction mixture was diluted with ethyl acetate, and the organic solution was washed with brine and dried over anhydrous sodium sulfate. After filtration and condensation under vacuum, the crude product was purified through flash silica gel column chromatography (hexane/acetone, 9/1 to 7/1) to give desired 3-(tert-butyldiphenylsilanyloxy)-1-(4-nitrophenyl)-propan-1-ol product (1.06 g, 95%). NMR (300 MHz, CDCl₃), 8.20 (d, J=8.1 Hz, 2H), 7.70-7.30 (m, 1H), 5.20-5.10 (m, 1H), 4.05 (br s, 1H, OH), 3.90-3.80 (m, 2H), 2.00-1.90 (m, 2H), 1.10 (s, 9H); IR (film) 3400, 2940, 2920, 1840, 1500, 1410, 1335, 1100, 685 cm⁻¹; MS (FAB⁺, 3NBA) m/z (relative intensity) 436.2 (M+1, 2.2), 418.1 (M-OH, 2.0), 378.1 (M-Bu, 1.5); HRMS (FAB⁺) calculated for C₂₅H₃₀NO₄Si (M+1) 436.1944, found 436.1932.

3-Amino 3-(4-nitrophenyl)-propan-1 ol. To the solution of 3-(tert-butyldiphenylsilanyloxy)-1-(4-nitrophenyl)-propan-1-ol (5.57 g, 12.8 mmol) in 50 mL of freshly distilled THF was added triphenyl phosphine (1.3 eq., 4.36 g). After cooling in ice-water bath for a few minutes, diethyl azodicarboxylate (1.3 eq., 2.89 g) and hydrazoic acid solution (1.2 M in THF, 2.4 eq., 21 mL) were added. The reaction was stirred at ambient temperature for 5 hours and quenched by saturated sodium bicarbonate. Ethyl ether extracted the mixture and the organic layer was washed by brine. After drying over anhydrous magnesium sulfate and condensation under vacuum, the crude product was purified through flash silica gel column chromatography to afford the desired 3-(tert-butyldiphenylsilanyloxy)-1-(4-nitrophenyl)-propyl azide intermediate (5.69 g, 97%). NMR (300 MHz, CDCl₃) δ 8.14 (d, J=8.7 Hz, 2H), 7.66-7.53 (m, 5H), 7.39-7.28 (m, 7H), 4.84 (dd, J=6.3, 8.1 Hz, 1H), 3.80-3.70 (m, 1H), 3.56-3.50 (m, 1H), 1.92-1.83 (m, 2H), 1.04 (s, 9H); IR (film) 2890, 2820, 2070, 1500, 1405, 1325, 1235, 1080, 775, 675 cm⁻¹; MS (FAB⁺, 3NBA) m/z (relative intensity) 461.3 (M+1, 2.1), 419.3 (3.6), 403.2 (M-Bu, 17.1).

The azide intermediate (300 mg, 0.66 mmol) was dissolved in 6 mL of anhydrous methanol. To the solution were added 0.33 mL (3.28 mmol, 5 eq.) of propane-1,3-dithiol and 0.46 mL (3.28 mmol, 5 eq.) of triethylamine. The reaction solution was allowed to stir at room temperature for 12 hours. The solvent was removed under reduced pressure. The residue was subject to flash silica gel column chromatography (chloroform/methanol, 30/1) to give the corresponding amine intermediate as a yellow oil (198 mg, 70%). NMR (300 MHz, CDCl₃) δ 8.15 (dd, J=2.1, 6.6 Hz, 2H), 7.68-7.36 (m, 12H), 4.33 (t, J=6.8 Hz, 1H), 3.76-3.64 (m, 2H), 1.95-1.80 (m, 2H), 1.72 (br s, 2H, NH), 1.08 (s, 9H); IR (film) 3040, 2920, 2840, 1650, 1585, 1500, 1410, 1325, 1080, 835, 805, 720, 680 cm⁻¹; MS (FAB⁺, 3NBA) m/z (relative intensity) 435.2 (M+1, 35.3), 377.1 (M Bu, 13.1), 257.1 (M-Ph, 5.8); HRMS (FAB⁺) calc'd for C₂₅H₃₁N₂₀O₃Si (M+1) 435.2104, found 435.2119.

At 0° C., 2.3 mL (2.3 mmol, 5 eq.) of 1 M of tetrabutylamonium fluoride solution in THF was added dropwise to the solution of amine intermediate (200 mg, 0.46 mmol). Subsequently, the reaction mixture was allowed to stir at room temperature for 1 hours, after which saturated aqueous potassium hydrosulfate was added to acidify the solution. After washing with ethyl ether, the aqueous solution was basified with 3 N of sodium hydroxide and extracted with methylene chloride (40 ml×3). The combined organic phase was dried over sodium sulfate. After filtration and concentration under reduced pressure, the residue was subjected to flash silica gel column chromatography (chloroform/methanol, 50/1 to 40/1, the chloroform was saturated with ammonium hydroxide) to afford the desired 3-amino-3-(4-nitrophenyl)-propan-1-ol as a white solid (74 mg, 82%). NMR (300 MHz, CDCl₃) □ 8.23 (d, J=9.0 Hz, 2H), 7.51 (d, J=9.0 Hz, 2H), 4.34-4.25 (m, 1H), 3.81 (t, J=5.25 Hz, 2H), 2.16 (br s, 3H), 1.95-1.89 (m, 2H); IR (film) 3300, 2900, 1580, 1495, 1330, 1040, 835, 730, 680 cm⁻¹; MS (FAB⁺, 3NBA) m/z (relative intensity) 197.1 (M+1, 100.00), 180.1 (M-OH, 13.9), 181.1 (M−NH₂, 9.8); HRMS (FAB⁺) calc'd for C₉H₁₃N₂O₃ (M+1) 197.0926, found 197.0946.

2-[Bis(2-chloroethyl)amino]-4 (p-nitrophenyl)-2H-1,3,2-oxazaphosphorinane 2-oxide (13b). 3-Amino-3-(4 nitrophenyl)-propan-1-ol (65 mg, 0.33 mmol) was dissolved in 40 mL of anhydrous ethyl acetate and cooled to 0° C. To the solution was added triethylamine (111 μL, 2.4 eq.) and a solution of bis(2-chloroethyl)phosphoramidic dichloride (103 mg, 1.2 eq.) in 10 mL of ethyl acetate. The reaction mixture was then allowed to stir at room temperature for 46 hours. After filtration to remove the white precipitate, the filtrate was washed with brine and dried over sodium sulfate. Filtration and concentration to remove organic solvent followed by flash silica gel column chromatography (petroleum ether/ethyl acetate, 1/3 for cis, chloroform/methanol=30/1 for trans) afforded two diastereomers: cis-13b (17 mg, 13.5%) and trans-13b (21.3 mg, 16.9%).

cis-13b: NMR (300 MHz, CDCl₃) δ 8.16 (d, J=9.0 Hz, 2H), 7.71 (d, J=9.0 Hz, 2H), 4.74 (t, J=7.2 Hz, 1H), 4.31-4.16 (m, 2H), 3.64-3.33 (m, 8H), 3.09 (d, J=3.6 Hz, 1H), 2.26-2.21 (m, 1H), 2.04-1.96 (m, 1H); ³¹P NMR (300 MHz, CDCl₃) δ 9.64 (s); IR (film) 3350, 3180, 2900, 1700, 1585, 1500, 1325, 1210, 1110, 1090, 970, 930, 840, 720, 680 cm⁻¹; MS (FAB⁺, 3NBA) m/z (relative intensity) 386.0 (M+5, 3.9), 384.0 (M+3, 38.8), 382.0 (M+1, 56.7), 346.1 (M-Cl, 5.6); HRMS (FAB⁺) calc'd for C₁₃H₁₉N₃O₄PCl₂ (M+1) 382.0490, found 382.0491; calc'd for C₁₃H₂₁N₃O₄P³⁵Cl³⁷Cl (M+3) 384.0461, found 384.0467.

trans-13b: m.p. (CHCl₃-MeOH) 139.5-141° C.; NMR (300 MHz, CDCl₃) δ 8.24 (d, J=8.1 Hz, 2H), 7.54 (d, J=8.1 Hz, 2H), 4.81 (dd, J=4.8, 9.9 Hz, 1H), 4.65-4.56 (m, 1H), 4.38-4.23 (m, 1H), 3.70-3.48 (m, 8H), 2.85 (br s, 1H, NH), 2.00-1.92 (m, 2H); 31P NMR (300 MHz, CDCl₃) δ 14.21 (s); IR (KBr) 3447, 3112, 2995, 2876, 1521, 1449, 1349, 1222, 1193, 1109, 914, 874, 750 cm⁻¹; MS (FAB, 3NBA) m/z (relative intensity) 386.1 (M+5, 6.1), 384.0 (M+3, 42.9), 382.0 (M+1, 65.9); HRMS (FAB⁺) calc'd for C₁₃H₁₉N₃O₄PCl₂ (M+1) 382.0490, found 382.0464; calc'd for C₁₃H₂₁N₃O₄P³⁵Cl³⁷Cl (M+3) 384.0461, found 384.0440.

### EXAMPLE 8

### Synthesis of 2-[Bis(2-chloroethyl)amino]-6-(p-nitrophenyl)-2H-1,3,2-oxazaphosphorinane 2-oxide (13c)

For the synthesis of 6-(p-nitrophenyl) cyclophosphamide (13c), the secondary hydroxyl group in 1-(4-nitrophenyl)-prop-2-en-1-ol was MOM-protected before hydroboration was performed. After hydroboration, to give 3-methoxymethoxy-3-(4-nitrophenyl)-propan-1-ol, the primary hydroxyl group was converted to amino group using a three step, activation by MsCl, S_{N}2 replacement using sodium azide, and triphenyl phosphine-mediated reduction. Catechol borane bromide (CBB) treatment followed by the addition of 1 equivalent of acetic acid removed the MOM protection group to give the 3-amino-1-(p-nitrophenyl)-1-propanol. Final cyclization of the 1,3-aminoalcohol with bis(2-chloroethyl)phosphoramidic dichloride gave the desired product 13c. Two diastereomers were separated using silica gel chromatography.

3-Methoxymethoxy-3-(4-nitrophenyl)-propan-1-ol. A solution of 1-(4-nitrophenyl)-prop-2-en-1-ol (1.94 g, 10.8 mmol) in 40 mL of dry dichloromethane was cooled in ice water bath for 15 minutes and treated sequentially with diisopropylethylamine (11.33 mL, 6 eq.) and chloromethyl methyl ether (4.94 mL, 6 eq.). The reaction mixture was stirred at ambient temperature for 24 hours before quenching with 5% sodium bicarbonate and extraction with ethyl ether. The organic extract was washed with brine and dried over anhydrous magnesium sulfate. After filtration and concentration under reduced pressure, the crude product was purified through flash silica gel column chromatography (hexane/ethyl acetate, 8/1 to 6/1) to give the MOM-protected intermediate (2.31 g, 95%). NMR (300 MHz, CDCl₃) δ 8.10 (dd, J=1.8, 6.9 Hz, 2H), 7.54-7.51 (m, 2H), 5.90-5.78 (m, 1H), 5.39-5.27 (m, 2H), 5.18 (d, J=6.6 Hz, 1H), 4.78 (d, J=4.8 Hz, 1H), 4.61 (d, J=5.7 Hz, 1H), 3.36 (s, 3H); IR (film) 3020, 2920, 2880, 1580, 1500, 1330, 1130, 1080, 1020, 900, 835 cm⁻¹; MS (FAB⁺, 3NBA) in/z (relative intensity) 224.1 (M+1, 22.4), 194.1 (M-30, 1.5), 208.1 (M-Me, 3.1), 192.1 (M-OMe, 1.3), 162.1 (M-OMOM, 77.5); HRMS (FAB⁺) calc'd for C₁₁H₁₄NO₄ (M+1) 224.0923, found 224.0924.

A solution of the MOM-protected intermediate (742 mg, 3.33 mmol) in 15 mL of dry THF was cooled in ice-water bath for several minutes and charged with a borane solution (1M, 1 eq., 3.3 mL). The reaction mixture was stirred at 0° C. for 5 hours and quenched slowly with 3 N sodium hydroxide (3.5 mL) and 30% hydrogen peroxide (3.5 mL). After another 30 minutes, ethyl acetate was added. The organic phase was washed with brine and dried over anhydrous sodium sulfate. After concentration under reduced pressure, the crude product was purified through flash silica gel column chromatography (hexane/ethyl acetate, 2/1 to 1/1) to afford the desired 3-methoxymethoxy-3-(4-nitrophenyl)-propan-1-ol product (625 mg, 78%). NMR (300 MHz, CDCl₃) □ 8.21 (dd, J=1.8, 6.8 Hz, 2H), 7.53-7.50 (m, 2H), 4.95 (dd, J=4.5, 8.9 Hz, 1H), 4.62 (d, J=6.6 Hz, 1H), 4.52 (d, J=6.9 Hz, 1H), 3.83-3.77 (m, 1H), 3.75-3.71 (m, 1H), 3.38 (s, 3H), 2.21 (br s, 1H, OH), 2.04-1.92 (m, 2H); IR (film) 3400, 2950, 1500, 1330, 1130, 1080, 1010 cm⁻¹; MS (FAB⁺, 3NBA) m/z (relative intensity) 242.1 (M+1, 19.6), 210.1 (M-OMe, 25.8), 224.1 (M-OH, 5.6); HRMS (FAB⁺) calc'd for C₁₁H₁₆NO₅ (M+1) 242.1028, found 242.1030.

3-Amino 1-(4-nitrophenyl)-propan-1 ol. A solution of 3-methoxymethoxy-3-(4-nitrophenyl)-propan-1-ol (128 mg, 0.53 mmol) in 10 mL of dry methylene chloride was cooled in ice-water bath for several minutes and then treated with triethyl amine (0.22 mL, 3 eq.) and methanesulfonyl chloride (80 μL, 2eq.). After stirring for 15 minutes, the reaction mixture was diluted with ether. The organic phase was washed with saturated sodium bicarbonate and brine, and was dried over anhydrous magnesium sulfate. After concentration under reduced pressure, the crude product was dissolved in 10 mL of dry DMF. To the solution was then added sodium azide (207 mg, 6 eq.) and 15-crown-5. The reaction was stirred at ambient temperature for 4.5 hours and partitioned between ethyl ether and water. The organic layer was washed with brine and dried over anhydrous magnesium sulfate, and evaporated to remove solvent under reduced pressure. The crude product was purified through flash silica gel column chromatography (hexane/ethyl acetate, 4/1 to 3/1) to afford the corresponding compound (129 mg, 91%). NMR (300 MHz, CDCl₃) δ 8.22 (dd, J=1.8, 6.8 Hz, 2H), 7.52 (dd, J=0.3, 6.9 Hz, 2H), 4.83 (dd, J=4.5, 9.0 Hz, 1H), 4.60 (d, J=6.9 Hz, 1H), 4.51 (d, J=1.2, 6.8 Hz, 1H), 3.51-3.40 (m, 2H), 3.37(s, 3H), 2.07-2.02 (m, 1H), 1.93-1.89 (m, 1H); IR (film) 2955, 2070, 1580, 1500, 1330, 1135, 1080, 1020 cm⁻¹; MS (FAB⁺, 3NBA) m/z (relative intensity) 267.2 (M+1, 4.8), 207.1 (3.6), 198.1 (5.6); HRMS (FAB⁺) calc'd for C₁₁H₁₅N₄O₄ (M+1) 267.1093, found 267.1082.

To a solution of the azide (4.13 g, 15.45 mmol), in 80 mL of THF (0.5% water), was added triphenyl phosphine (4.12 g, leg.). The reaction mixture was stirred at room temperature for 24 hours and was concentrated under reduced pressure. The crude product was purified through flash silica gel column chromatography to afford the desired MOM-protected amino alcohol (2.69 g, 72%). NMR (300 MHz, CDCl₃) δ 8.21 (dd, J=2.1, 6.9 Hz, 2H), 7.50 (d, J=8.7 Hz, 2H), 4.83 (dd, J=4.8, 8.4 Hz, 1H), 4.59 (d, J=6.9 Hz, 1H), 4.50 (dd, J=0.3, 6.9 Hz, 1H), 3.37 (s, 3H), 2.83 (t, J=6.9 Hz, 2H), 2.00-1.93 (m, 1H), 1.82-1.75 (m, 1H), 1.31 (br s, 2H, NH); IR (film) 2900, 1630, 1580, 1500, 1330, 1130, 1080, 1000, 900, 830, 680 cm⁻¹; MS (FAB⁺, 3NBA) m/z (relative intensity) 241.1 (M+1, 100.00), 225.1 (M-Me, 1.9), 209.1 (M-OMe, 1.5); HRMS (FAB⁺) calc'd for C₁₁H₁₇N₂01 (M+1) 241.1188, found 241.1182.

A solution of the above intermediate (1.0 g, 4.17 mmol) in 50 mL of dry dichloromethane was cooled under −50° C. and treated with B-bromocatecholborane solution (17 mL of 0.245 N in dichloromethane, 1 eq.). The reaction mixture was allowed to warm up to −20° C. for 2 hours and treated with glacial acid (0.24 mL, 1 eq.). After stirring at room temperature for another 7 hours, the reaction mixture was quenched with 3 N sodium hydroxide and extracted with chloroform. The organic layer was washed with brine and dried over anhydrous magnesium sulfate. After concentration under reduced pressure, the crude product was purified through flash silica gel column chromatography (chloroform/methanol, 9/1 to 8/1) to afford the desired 3-amino-1-(4-nitrophenyl)-propan-1-ol product (629 mg, 77w) m.p. (CHCl₃-MeOH): 126-127.5° C.; NMR (300 MHz, CDCl₃) δ 8.13 (dd, J=2.0, 6.9 Hz, 2H), 7.52-7.47 (m, 2H), 5.03 (dd, J=2.7, 8.7 Hz, 1H), 3.12-3.06 (m, 1H), 3.07-2.92 (m, 1H), 1.99-1.81 (m, 1H), 1.67-1.41 (m, 1H); IR (KBr) 3330, 3260, 3100, 2880, 2850, 1575, 1490, 1400, 1330, 1300, 1275, 1075, 1085, 1050, 1000, 935, 810, 730, 680 cm⁻¹; MS (FAB⁺, 3NBA) m/z (relative intensity) 197.1 (M+1, 30.5), 181.0 (M-OH, 1.8); HRMS (FAB⁺) calc'd for C₉H₁₃N₂O₃ (M+1) 197.0926, found 197.0939.

2-[Bis(2-chloroethyl)amino]-6-(p-nitrophenyl)-2H-1,3,2 oxazaphosphorinane 2-oxide (13c). A solution of 3-amino-1-(4-nitrophenyl)-propan-1-ol (131 mg, 0.67 mmol) in 20 mL of ethyl acetate was cooled in ice-water bath for several minutes and treated with Et₃N (185 μL, 2 eq.) and a solution of bis(2-chloroethyl)phosphoramidic dichloride (173 mg, 1 eq.) in 5 mL of ethyl acetate. The reaction mixture was stirred at room temperature for 24 hours and partitioned between ethyl acetate and water. The organic phase was washed with brine and dried over anhydrous sodium sulfate. After filtration and concentration under reduced pressure, the crude product was purified through flash column silica gel chromatography (chloroform/methanol, 30/1 to 15/1) to afford two diastereomers: cis-13c (79 mg, 33.5%) and trans-13c (77 mg, 32.5%).

cis-13c: m.p. (CHCl₃-MeOH) 125-127° C.; NMR (300 MHz, CDCl₃) δ 8.21 (dd, J=1.8, 6.9 Hz, 2H), 7.62 (d, J=8.7 Hz, 2H), 5.50-5.40 (m, 1H), 3.80-3.60 (m, 6H), 3.52-3.35 (m, 5H), 2.20-1.95 (m, 2H); 31P NMR (300 MHz, CDCl₃) δ 11.18 (s); IR (KBr) 3400, 3140, 2920, 2820, 1580, 1490, 1420, 1325, 1220, 1195, 1095, 1075, 1020, 965, 890, 840, 830, 790, 725 cm⁻¹; MS (FAB⁺, 3NBA) m/z (relative intensity) 384.2 (M+3, 2.9), 382.2 (M+1, 4.1); HRMS (FAB⁺) calc'd for C₁₃H₁₉N₃O₄PCl₂ (M+1) 382.0490, found 382.0479; calc'd for C₁₃H₁₉N₃O₄P³⁵Cl³⁷Cl (M+3) 384.0461, found 384.0459.

trans-13c: m.p. (CHCl₃-MeOH) 138-140° C.; NMR (300 MHz, CDCl₃) δ 8.20 (dd, J 1.8, 6.8 Hz, 2H), 7.50 (d, J=9.6 Hz, 2H), 5.63 (d, J=11.1 Hz, 1H), 3.65-3.30 (m, 10H), 3.10 (br s, 1H, NH), 2.10-1.80 (m, 2H); ³¹p NMR (300 MHz, CDCl₃) δ 14.58 (s); IR (KBr) 3400, 3120, 2920, 2760, 1590, 1500, 1435, 1330, 1200, 1080, 940, 900, 730 cm⁻¹; MS (FAB⁺, 3NBA) m/z (relative intensity) 384.0 (M+3, 1.7), 382.1 (M+1, 3.3); HRMS (FAB⁺) calc'd for C₁₃H₁₉N₃O₄PCl₂ (M+1) 382.0490, found 382.0482; calc'd for C₁₃H₁₉N₃O₄P³⁵Cl³⁷Cl (M+3) 384.0461, found 384.0462.

### EXAMPLE 9

### Synthesis of 2-[Bis(2-chloroethyl)amino]-4-(p-nitrophenyl)-2H-1,3,2-diazaphosphorinane 2-oxide (13d)

The diaza cyclophosphamide analogue 13d was synthesized by converting 1-p-nitrophenyl-1,3-propane-diol, using a Mitsunobu reaction, to the corresponding diazido followed by 1,3-propanedithiol reduction and cyclization of the resulting diamine with bis(2-chloroethyl)phosphoramidic dichloride. Two diastereomers were separated using silica gel chromatography.

1-(4-Nitro-phenyl)-propane-1,3-diazide. To a solution of 1-p-nitrophenyl-1,3-propane-diol (709 mg, 3.6 mmol) and triphenylphosphine (2.83 g, 10.8 mmol, 1.5 eq.) in 50 mL of anhydrous THF was added, at room temperature, a hydrazoic acid solution (18 mL of 1.2 M in benzene) and subsequently a solution of diethyl azodicarboxylate (1.68 mL, 10.8 mmol, 1.5 eq.) dissolved in 10 mL of anhydrous THF. The reaction mixture was stirred at room temperature for 12 hours. Brine was added to quench the reaction. The solution was extracted with 100 mL of ethyl ether. The organic phase was washed with saturated aqueous sodium bicarbonate solution and brine, dried over anhydrous sodium sulfate. After filtration and concentration, the residue was subjected to flash silica gel column chromatography (hexane/ethyl acetate: 6/1) to afford the desired 1-(4-nitro-phenyl)-propane-1,3-diazide as an oil (653 mg, 73.4%). ¹H NMR (300 MHz, CDCl₃) δ 8.27 (d, J=8.1 Hz, 2H), 7.52 (d, J=8.4 Hz, 2H), 4.81-4.70 (m, 1H), 3.57-3.42 (m, 1H), 3.40-3.30 (m, 1H), 2.05-1.82 (m, 2H); IR (film): 2980, 2080, 1720, 1510, 1470, 1425, 1335, 1220, 1170, 1110, 1050, 980, 700, 680 cm⁻¹; MS (FAB, 3NBA) m/z (relative intensity) 248.1 (M+1, 7.9), 219.2 (M−28, 30.8), 177.1(31.5).

1-(4-Nitrophenyl)-propane-1,3-diamine. 1-(4-Nitro-phenyl)-propane-1,3-diazide (625 mg, 2.53 mmol) was dissolved in 30 mL of anhydrous methanol. To the solution was added 1.0 mL (10 mmol, 2 eq.) of propane-1,3-dithiol and 1.4 mL (10 mmol, 2 eq.) of triethylamine. The reaction mixture was stirred at room temperature for 36 hours. The reaction mixture was filtered to remove the white precipitate. After concentration under reduced pressure, the crude product was purified through flash silica gel column chromatography (chloroform/methanol: 5/1 to 2/1, the chloroform was saturated with aqueous ammonia) to afford the desired 1-(4-nitro-phenyl)-propane-1,3-diamine as a reddish oil (428 mg, 87%). ¹H NMR (300 MHz, CDCl₃) δ 8.21 (d, J=9.0 Hz, 2H), 7.51 (d, J=9.0 Hz, 2H), 4.19 (t, J=6.80 Hz, 1H), 2.75 (t, J=6.75 Hz, 2H), 1.81-1.76 (m, 2H), 1.40 (br s, 4H, NH); IR (film) 3300, 2950, 1590, 1500, 1330, 840 cm⁻¹; MS (FAB⁺, 3NBA) m/z (relative intensity) 196.1 (M+1, 72.8), 165.1 (M-30, 8.4), 151.1 (14.2). HRMS (FAB⁺) m/z calc'd for C₉H₁₄N₃O₂ (MH⁺) 196.1086, found 196.1124.

2 [Bis(2 chloroethyl)amino]-4-(p-nitrophenyl)-2H-1,3,2-diazaphosphorinane 2-oxide (13d). 1-(4-Nitro-phenyl)-propane-1,3-diamine (50 mg, 0.26 mmol) was dissolved in 40 mL of ethyl acetate. To the solution was added 86 μL (0.61 mmol, 2.4 eq.) of triethylamine. After lowering the temperature to 0° C., a solution of bis(2-chloroethyl)phosphoramidic dichloride (78 mg, 0.3 mmol, 1.2 eq.) in 10 mL of ethyl acetate was added. The reaction mixture was stirred at room temperature for 39.5 hours. After removal of the white precipitate via filtration, the filtrate was washed with brine and dried over anhydrous sodium sulfate. Removal of organic solvent gave a crude product that was subjected to flash silica gel column chromatography (chloroform/methanol: 30/1 to 20/1) to give two chromatographically separable isomers: cis-13d (27 mg, 28%) and trans-13d (33 mg, 34%).

cis-13d: m.p. (CHCl₃-MeOH) 119-120° C.; ¹H NMR (300 MHz, CDCl₃) δ 8.22 (d, J=9.0 Hz, 2H), 7.69 (d, J=8.7 Hz, 2H), 4.70-4.60 (m, 1H), 3.69 (t, J=6.3 Hz, 4H), 3.60-3.20 (m, 8H), 2.15-2.00 (m, 1H), 1.90-1.80 (m, 1H); 31P NMR (300 MHz, CDCl₃) δ 12.91 (s); IR (KBr) 3140, 2940, 2900, 2830, 1580, 1495, 1440, 1330, 1190, 1155, 1100, 960, 890, 710 cm⁻¹; MS (FAB⁺, 3NBA) m/z (relative intensity) 381.1 (M+1, 72.4), 383.1 (M+3, 43.8), 385.1 (M+5, 9.4); HRMS (FAB⁺) m/z calc'd for C₁₃H₂₀N₄O₃PCl₂ (M+1) 381.0650, found 381.0626; calc'd for C₁₃H₂₀N₄O₃P³⁵Cl³⁷Cl (M+3) 383.0621, found 383.0630.

trans-13d: m.p. (CHCl₃-MeOH) 148.5-149.5° C.; ¹H NMR (300 MHz, CDCl₃) δ 8.24 (d, J=8.7 Hz, 2H), 7.54 (d, J=8.8 Hz, 2H) 4.78-4.70 (m, 1H), 3.70-3.50 (m, 12H), 2.10-1.75 (m, 2H); ³¹p NMR (300 MHz, CDCl₃) δ 17.09 (s); IR (KBr) 3140, 2900, 1570, 1500, 1450, 1430, 1410, 1325, 1190, 1150, 1090, 980, 850, 720 cm⁻¹; MS (FAB⁺, 3NBA) m/z (relative intensity) 385.1 (M+5, 1.0), 383.1 (M+3, 2.6), 381.1 (M+1, 10.1); HRMS (FAB⁺) m/z calc'd for C₁₃H₂₀N₄O₃PCl₂ (M+1) 381.0650, found 383.0643; calc'd for C₁₃H₂₀N₄O₃P³⁵Cl³⁷Cl (M+3) 383.0621, found 383.0612.

### EXAMPLE 10

### Method of Synthesizing Nitrobenzyl N,N-bis(2-chloroehtyl)phosphordiamidates (15)

A general synthesis for nitrobenzyl N,N-bis(2-chloroehtyl)phosphordiamidates is as follows. Bis(2-chloroethyl)phosphoramidic dichloride (1.4 g, 5.5 mmol) was dissolved in 20 mL of THF and cooled to −78° C. Meanwhile, a benzyl alcohol (5 mmol) was dissolved in 10 mL of THF, cooled to −78° C., and to it was slowly added a solution of butyl lithium in hexane (2.5 M, 2.2 mL, 5.5 mmol). The mixture was stirred at −78° C. for 10 minutes and subsequently added, with vigorous stirring at −78° C., to the above phosphoramidic dichloride solution via syringe. The resulting solution was kept at −78° C. for 2 hours. Ammonia gas was bubbled through the solution at a moderate rate for 30 minutes at −78° C. The THF was evaporated, and the resulting residue was partitioned between CH₂Cl₂ (30 mL) and water (30 mL). The two phases were separated and the aqueous phase was extracted with CH₂Cl₂ (2×20 mL). The combined organic phase was washed with saturated NaCl solution (2×20 mL) and dried over Na₂SO₄. After evaporation, the residue was purified by flash silica gel column chromatography (CH₂Cl₂/CH₃OH, 30:1) afforded the desired product.

2-Nitrobenzyl N,N-bis(2-chloroethyl) phosphor-diamidate (15a). Using the general synthesis scheme starting from 2-nitrobenzyl alcohol (765 mg, 5 mmol) afforded the desired product as a yellow solid (530 mg, 30%). m.p. 69-71° C.; ¹H NMR (CD₃OD, 200 MHz) δ 3.42-3.54 (m, 4H), 3.66-3.73 (m, 4H), 5.42 (d, 2H, J=7.0 Hz), 7.59 (dt, 1H, J=1.2, 8.1 Hz), 7.79 (dt, 1H, J=1.2, 7.3 Hz), 7.88 (d, 1H, J=7.4 Hz), 8.16 (dd, 1H, J=1.2, 8.0 Hz); ¹³C NMR (CD₃OD, 50 MHz) δ 16.6, 29.8, 63.1, 63.2, 123.9, 127.9, 128.0, 132.4, 132.6, 133.1; MS (ESI⁺) m/z (relative intensity): 221 (17), 262 (10), 356.0 (MH⁺, 100), 358 (MH⁺+2, 70), 370 (MH⁺+4, 12), 378 (M+Na⁺, 10).

3-Nitrobenzyl N,N-bis(2-chloroethyl) phosphor diamidate (15b). Using the general synthesis scheme starting from 3-nitrobenzyl alcohol (765 mg, 5 mmol) afforded the desired product as a yellow solid (930 mg, 53%). m.p. 92-93° C.; ¹H NMR (CD₃OD, 200 MHz) δ 3.40-3.53 (m, 4H), 3.65-3.73 (m, 4H), 5.13 (d, 2H, J=7.6 Hz), 7.66 (t, 1H, J=1.2, 8.0 Hz), 7.84 (dd, 1H, J=1.2, 6.8 Hz), 8.23 (d, 1H, J=8.2 Hz), 8.34 (s, 1H, J=8.2 Hz); ¹³C NMR (CD₃OD, 50 MHz) δ 41.1, 64.8, 64.9, 121.2, 122.0, 128.9, 132.6; MS (ESI⁺) m/z (relative intensity): 356.0 (MH⁺, 100%), 358 (MH⁺+2, 70), 360 (MH⁺+4, 12), 397 (MH⁺+41, 41), 399 (30), 401 (4).

4-Nitrobenzyl N,N-bis(2-chloroethyl) phosphor-diamidate (15c). Using the general synthesis scheme starting from 4-nitrobenzyl alcohol (765 mg, 5 mmol) afforded the desired product as a light yellow solid (940 mg, 53%). m.p. 86-88° C.; ¹H NMR (CD₃OD, 200 MHz) δ 3.41-3.53 (m, 4H), 3.66-3.73 (m, 4H), 3.99 (s, 3H), 5.14 (d, 2H, J=7.4 Hz), 7,67 (d, 1H, J=7.6 Hz), 8.26 (d, 2H, J=7.0 Hz). ¹³C NMR (CD₃OD, 50 MHz) δ 41.2, 64.8, 64.9, 122.7, 127.1, 144.0, 144.1, 147.1. MS (ESI⁺) m/z (relative intensity): 356 (MH⁺, 100%), 358 (MH⁺+2, 70), 360 (MH⁺+4, 12), 397 (MH⁺+41, 34), 399 (20), 401 (2).

1 (4-Nitrophenyl)ethyl N,N bis(2-chloroethyl)phos -phordiamidate (15d). To a solution of 4-nitroacetophenone (500 mg, 3 mmol) in 3 mL of ethanol and 3 mL of THF at 5° C. were added, with stirring, sodium borohydride (168 mg, 4.4 mmol) and 2 N sodium hydroxide (2.4 mL). The solution was allowed to come to room temperature and stirred for 2 hours and subsequently quenched with 2 N hydrochloric acid to pH=6. After dilution with water, the reaction solution was extracted with CH₂Cl₂ (2×20 mL). The combined organic phase was washed with saturated NaCl solution (3×30 mL) and dried over Na₂SO₄. After evaporation, the residue was purified by flash silica gel column chromatography (hexanes/EtOAc, 5:1) to afford the desired 1-(4-nitrophenyl)ethanol as a light yellow oil (434 mg, 869%). ¹H NMR (CDCl₃, 200 MHz) δ 1.55 (d, 3H, J=6.6 Hz), 2.01 (s, 1H), 5.05 (q, 1H, J=6.6 Hz), 7.54-7.60 (m, 2H), 8.20-8.26 (m, 2H).

Using the general synthesis scheme starting from 1-(4-nitrophenyl)ethanol (200 mg, 1.2 mmol) described herein afforded two chromatographically separable diastereomers (a less polar isomer A as a light foam solid, 56 mg and a more polar isomer B as a light yellow oil, 62 mg, combined 32%).

Isomer A: ¹H NMR (CD₃OD, 200 MHz) δ 1.63 (d, 3H, J=6.6 Hz), 3.40-3.54 (m, 4H), 3.68-3.75 (m, 4H), 5.54-5.61 (m, 2H), 7.64-7.70 (m, 2H), 8.22-8.29 (m, 2H); ¹³C NMR (CD₃OD, 50 MHz) δ 23.1, 23.2, 41.2, 72.9, 73.0, 122.8, 125.9, 147.0, 149.7; MS (EST+) m/z (relative intensity): 221 (26%), 262(100), 370 (MH⁺, 60), 372 (MH⁺+2, 42), 360 (MH⁺4, 4), 411(7).

Isomer B: ¹H NMR (CD₃OD, 200 MHz) δ 1.63 (d, 3H, J=6.6 Hz), 3.22-3.36 (m, 4H), 3.50-3.62 (m, 4H), 5.52-5.60 (m, 2H), 7.65-7.70 (m, 2H), 8.24-8.30 (m, 2H); ¹³C NMR (CD₃OD, 50 MHz,) δ 23.1, 23.2, 41.1, 72.8, 72.9, 122.9, 126.0, 147.0, 149.5; MS (ESI⁺) m/z (relative intensity): 221 (100%), 262 (33), 370 (MH⁺, 33), 372 (MH⁺+2, 18), 360 (MH⁺+4, 1).

3-Carboxamide-4 nitrobenzyl N,N-bis(2-chloroethyl) phosphordiamidate (15e). 3-Methoxycarbonyl-4-nitrobenzyl alcohol (290 mg, 1.4 mmol) was suspended in 4 mL of saturated ammonia in methanol. The solution was heated to 60° C. for 6 days. The solvent was evaporated and the residue was purified by flash silica gel column chromatography to afford 5-hydroxymethyl-2-nitrobenzamide as a white solid (196 mg, 73%). m.p. 143-145° C.; ¹H NMR (CD₃OD, 200 MHz) δ 3.33 (s, 2H), 4.75 (s, 2H), 7.60 (s, 1H), 7.63 (d, 1H, J=8.4 Hz), 8.08 (d, 1H, J=8.0 Hz).

Using the general synthesis scheme starting from 5-hydroxymethyl-2-nitrobenzamide (91 mg, 0.51 mmol) afforded the desired product as an oil (11 mg, 6%). ¹H NMR (CD₃OD, 200 MHz) δ 3.41-3.54 (m, 4H), 3.67-3.74 (m, 4H), 5.14 (d, 2H, J=7.2 Hz), 7.70 (d, 1H, J=8.4 Hz), 7.73 (s, 1H), 8.11 (s, 1H, J=8.4 Hz); ¹³C NMR (CD₃OD, 50 MHz) δ 41.2, 41.2, 64.4, 64.5, 123.8, 126.3, 127.2, 128.0, 128.0, 132.2, 143.0, 143.2; MS (ESI⁺) m/z (relative intensity): 399 (MH⁺, 100%), 401 (MH⁺+2, 67), 403 (MH⁺+4, 12).

3-Methoxycarbonyl-4-nitrobenzyl N,N-bis(2-chloroethyl) phosphordiamidate (15f). 5-Methyl-2-nitrobenzoic acid (3.62 g, 20 mmol) was dissolved in 50 mL of methanol. After the addition of several drops of concentrated sulfuric acid, the reaction mixture was heated to reflux for 48 hours. The solvent was evaporated, and the resulting residue was partitioned between CH₂Cl₂ (30 mL) and water (30 mL). The two phases were separated and the aqueous phase was extracted with CH₂Cl₂ (2×20 mL). The combined organic phase was washed with saturated NaCl solution (2×20 mL) and dried over Na₂SO₄. After evaporation, the residue was purified by flash silica gel column chromatography (hexanes/EtOAc, 8:1) to afford 5-methyl-2-nitrobenzoic acid methyl ester as a light yellow solid (3.08 g, 79%). m.p. 78-79 C; ¹H NMR (CDCl₃, 200 MHz) δ 2.50 (s, 3H), 3.94 (s, 2H), 7.40 (ddd, 1H, J=0.8, 1.8, 10 Hz), 7.50 (s, 1H), 7.90 (d, 1H, J=8.4 Hz).

5-Methyl-2-nitro-benzoic acid methyl ester (1.6 g, 8 mmol) and bromosuccinimide (1.75 g, 9.8 mmol) were suspended in 80 mL of CCl₄. The solution was photolyzed overnight with a 300 watt lamp. The reaction solution was washed with saturated NaCl solution (3×20 mL) and dried over anhydrous Na₂SO₄. After evaporation, the residue was purified by flash silica gel column chromatography (hexanes/EtOAc, 10:1) to afford 3-methoxycarbonyl 4-nitrobenzyl bromide as a yellow solid (530 mg, 69%). m.p. 55-56° C.; ¹H NMR (CDCl₃, 200 MHz) δ 3.95 (s, 3H), 4.52 (s, 2H), 7.66 (dd, 1H, J=1.8, 8.4 Hz), 7.76 (d, 1H, J=2.2 Hz), 7.92 (d, 1H, J=8.4 Hz).

3-Methoxycarbonyl-4-nitrobenzyl bromide (530 mg, 1.9 mmol) and CaCO₃ (1.16 g, 11.6 mmol) were suspended in dioxane and H₂O mixture and the solution was heated to reflux overnight. The solvent was evaporated, and the resulting residue was partitioned between CH₂Cl₂ (30 mL) and water (30 mL). The two phases were separated and the aqueous phase was extracted with CH₂Cl₂ (2×20 mL) The combined organic phase was washed with saturated NaCl solution (2×20 mL) and dried over Na₂SO₄. After evaporation, the residue was purified by flash silica gel column chromatography (hexanes/EtOAc, 8:1) to afford 3 methoxycarbonyl-4-nitrobenzyl alcohol as a yellow solid (191 mg, 47%). m.p. 56-58° C.; ¹H NMR (CDCl₃, 200 MHz) δ 2.45 (s, 1H), 3.93 (s, 3H), 4.82 (s, 2H), 7.60 (dd, 1H, J=1.8, 8.4H)z, 7.68 (d, 1H, J=2.0 Hz), 7.97 (d, 1H, J=8.4 Hz).

Using the general synthesis scheme starting from 3-methoxycarbonyl-4-nitrobenzyl alcohol (82 mg, 0.45 mmol) afforded the desired product as an oil (21 mg, 13%). ¹H NMR (CD₃OD, 200 MHz) δ 3.40-3.53 (m, 4H), 3.64-3.73 (m, 4H), 3.92 (s, 3H), 5.10 (d, 2H, J=7.6 Hz), 7.78 (d, 1H, J=8.0 Hz), 7.85 (s, 1H), 8.02 (d, 1H, J=8.4 Hz); ¹³C NMR (CD₃OD, 50 MHz) δ 41.2, 51.8, 64.3, 64.4, 123.5, 127.3, 129.5; MS (ESI⁺) m/z (relative intensity): 414 (MH⁺, 100%), 416 (MH⁺+2, 70), 418 (MH⁺+4, 12), 436 (M+Na⁺, 25), 438 (20), 440 (2).

3-Methyl-4-nitrobenzyl N,N-bis(2-chloroethyl)phos phordiamidate (15g). Using the general synthesis scheme starting from 3-methyl-4-nitrobenzyl alcohol (765 mg, 5 mmol) afforded the desired product as a yellow solid (780 mg, 43%). m.p. 64-66° C.; ¹H NMR (CD₃OD, 200 MHz) δ 3.40-3.53 (m, 4H), 3.65-3.73 (m, 4H), 5.07 (d, 2H, J=7.8 Hz), 7.46 (d, 1H, J=8.6 Hz), 7.50 (s, 1H), 7.99 (d, 1H, J=8.0 Hz); ¹³C NMR (CD₃OD, 50 MHz) δ 18.3, 41.2, 64.7, 64.8, 123.9, 124.7, 130.3, 132.9, 124.0, 142.2; MS (ESI⁺) m/z (relative intensity): 370 (MH⁺, 100%), 372 (MH⁺+2, 70), 374 (MH⁺+4, 11), 411 (MH⁺+41, 20), 413 (12), 415 (1).

3 Methoxy-4-nitrobenzyl N,N bis(2-chloroethyl)phos-phordiamidate (15h). Using the general synthesis scheme starting from 3-methoxy-4-nitrobenzyl alcohol (228 mg, 1.2 mmol) afforded the desired product as a dark yellow oil (195 mg, 41%). ¹H NMR (CD₃OD, 200 MHz) δ 3.40-3.54 (m, 4H) 3.66-3.74 (m, 4H), 3.99 (s, 3H), 5.08 (d, 2H, J=7.2 Hz), 7.11 (dd, 1H, J=1.4, 8.4 Hz), 7.34 (d, 1H, J=1.2 Hz), 7.83 (d, 1H, J=8 Hz); ¹³C NMR (CD₃OD, 50 MHz) δ 41.2, 41.2, 55.3, 64.9, 65.0, 111.4, 117.7, 124.6, 143.6, 143.7, 152.3; MS (ESI⁺) m/z (relative intensity): 386.0 (MH⁺, 100%), 388 (MH⁺+2, 70), 390 (MH⁺+4, 10), 427 (MH⁺+41, 32), 399 (21) 401 (3).

2-Methoxy-4 nitrobenzyl N,N-bis(2-chloroethyl)phos-phordiamidate (15i). 2-methy-5-nitrophenol (1.53 g, 10 mmol), anhydrous potassium carbonate (1.03 g, 7.5 mmol), and iodomethane (1.56 g, 11 mmol) were suspended in 20 mL of dry acetone and heated to reflux for 5 hours. Water (10 mL) was added and acetone was evaporated. The residue was extracted with CH₂Cl₂ (2×20 mL). The CH₂Cl₂ phase was washed with saturated NaCl solution (2×20 mL) and dried over Na₂SO₄. After evaporation, the residue was purified by flash silica gel column chromatography (hexanes/EtOAc, 10:1) to afford 1-methyl-2-methoxy-4-nitrobenzene as a light yellow solid (1.2 g, 72%). m.p. 72-73° C.; ¹H NMR (CDCl₃, 200 MHz) δ 2.32 (s, 3H), 3.94 (s, 2H), 7.29 (d, 1H, J=8.2 Hz), 7.68 (d, 1H, J=2.2 Hz), 7.79 (dd, 1H, J=2.2, 8.0 Hz).

1-Methyl-2-methoxy-4-nitrobenzene (1.2 g, 7.2 mmol) and bromosuccinimide (1.4 g, 7.8 mmol) were suspended in 80 mL of CCl₄. The solution was photolyzed overnight with a 300 watt lamp. The reaction solution was washed with saturated NaCl solution (3×20 mL) and dried over anhydrous Na₂SO₄. After evaporation, the residue was purified by flash silica gel column chromatography (hexanes/EtOAc, 10:1) to afford 2-methoxy-4-nitrobenzyl bromide as a light yellow oil (1.22 g, 69%). ¹H NMR (CDCl₃, 200 MHz) δ 4.02 (s, 3H), 4.56 (s, 2H), 7.51 (d, 1H, J=8.4 Hz), 7.75 (d, 1H, J=2.2 Hz), 7.84 (dd, 1H, J=2.2, 8.4 Hz).

2-Methoxy-4-nitrobenzyl bromide (1.22 g, 5 mmol) and CaCO₃ (3 g, 30 mmol) were suspended in dioxane and H₂O mixture and the solution was heated to reflux overnight. The solvent was evaporated, and the resulting residue was partitioned between CH₂Cl₂ (30 mL) and water (30 mL). The two phases were separated and the aqueous phase was extracted with CH₂Cl₂ (2×20 mL). The combined organic phase was washed with saturated NaCl solution (2×20 mL) and dried over Na₂SO₄. After evaporation, the residue was purified by flash silica gel column chromatography (hexanes/EtOAc, 8:1) to afford 2-methoxy-4-nitrobenzyl alcohol as a light yellow solid (430 mg, 47%). ¹H NMR (CDCl₃, 200 MHz) δ 2.26-2.29 (br s, 1H), 3.96 (s, 3H), 4.78 (s, 2H), 7.53 (dd, 1H, J=0.6, 8.4 Hz), 7.71 (d, 1H, J=2.2 Hz), 7.82 (dd, 1H, J=1,8, 8.2 Hz).

Using the general synthesis scheme starting from 2-methoxy-4-nitrobenzyl alcohol (100 mg, 0.54 mmol) described herein afforded the desired product as a yellow solid (118 mg, 32%). m.p. 102-104° C.; ¹H NMR (CD₃OD, 200 MHz) δ 3.41-3.51 (m, 4H), 3.53-3.73 (m, 4H), 3.99 (s, 3H), 5.10 (d, 2H, J=7.0 Hz), 7.67 (d, 1H, J=8.0 Hz), 7.83 (d, 1H, J=2.2 Hz), 7.90 (dd, 1H, J=2.2, 8.4 Hz); ¹³C NMR (CD₃OD, 50 MHz) δ 41.1, 54.7, 60.9, 61.0, 104.2, 114.7, 132.3, 148.2, 156.5; MS (ESI⁺) m/z (relative intensity): 387 (MH⁺, 100%), 389 (MH⁺+2, 70), 391 (MH⁺+4, 12).

### EXAMPLE 11

### Stability Test of Compounds in Aqueous Buffer

A 2 mg sample of each compound provided herein was dissolved in 2 mL of 50 mM sodium phosphate buffer (pH=7.40) containing 10% DMSO and incubated at 37° C. At different time intervals, aliquots were withdrawn and subjected to reversed-phase HPLC analysis (C₁₈ analytical column, gradient elution from 5%-80% acetonitrile containing 0.1% TFA at a flow rate of 1 mL/minute).

### EXAMPLE 12

### Enzyme Assays

Substrate (0.2 mM) was incubated with 1 mM of NADH at 37° C. in 10 mM phosphate buffer (pH 7.0) in a total volume of 250 μL. The reaction was initiated by the addition of 1.8 μg of E. coli nitroreductase. Aliquots were withdrawn and analyzed by HPLC. The half-life of reduction by E. coli nitroreductase was calculated based on the disappearance of the substrate.

The same assays were also performed using a spectrophotometric assay. When NADH, the reduced form, donates its 2 electrons to nitroaromatics for its reduction to its corresponding hydroxylamine, NAD+ is formed. Two NADH molecules are required to reduce one molecule of nitroaromatic to hydroxylamine. This process can be followed by measuring changes in UV absorption at 340 nM. NADH with its reduced pyridine ring absorbs light at 340 nm, while NAD+ has the oxidized ring normally found in pyridine and lacks absorbance at 340 nm. So as NADH is converted to NAD+ during the nitroreductase-catalyzed reaction, the absorbance at 340 nm decreases. Initial velocity was calculated based on the absorbance change at 340 nm in the first 10% of the reaction.

### EXAMPLE 13

### Cell Culture and Antiproliferative Assays In Vitro.

V79 Chinese hamster lung fibroblasts were grown in monolayer culture in DMEM containing 10% fetal calf serum and 4 mM glutamine. Cells were maintained in a humidified atmosphere at 37° C. with 5% CO₂ and subcultured twice, weekly by trypsinization. The V79 cells were transfected with a bicistronic vector encoding for the E. coli nitroreductase or the human quinone oxidoreductase protein and puromycin resistance protein as the selective marker. The positive clones were selected in growth medium containing 10 μg/mL puromycin and maintained under selective pressure. Cells expressing either E. coli nitroreductase (T116) or human quinone oxidoreductase NQO1 (hDT7) in exponential phase of growth were trypsinized, seeded in 96-well plates at a density of 1000 cells/well, and permitted to recover for 24 hours. F179 cells were transfected with vector only and were used as the controls. The medium was replaced with fresh medium containing co-substrate (100 μM). Serial dilutions of the drug solution were performed in situ and cells were then incubated with drug for 3 days at 37° C. The plates were fixed and stained with SRB before reading with optical absorption at 590 nm; results were expressed as a percentage of control growth. IC₅₀ values are the concentration required to reduce cell number to 50% of control and were obtained by interpolation.

SKOV3 human ovarian carcinoma cells were infected with a newly prepared batch of adenovirus expressing wild-type nitroreductase, using multiplicities of infection of 100 pfu/cell; and uninfected cells as control. Cells were plated in 96-well plates (15000 cells/well) and incubated for 2 days to allow for nitroreductase expression. Used medium was exchanged with fresh medium containing a range of prodrug concentrations with a maximum drug concentration of 1 mM. After 18 hours of incubation with the prodrugs, the medium was replaced with fresh medium. An MTT assay was performed 3 days after adding prodrug to assess cell viability.

What is claimed is:

1. A nitroaryl-substituted phosphoramide compound comprising Formula I or Formula II 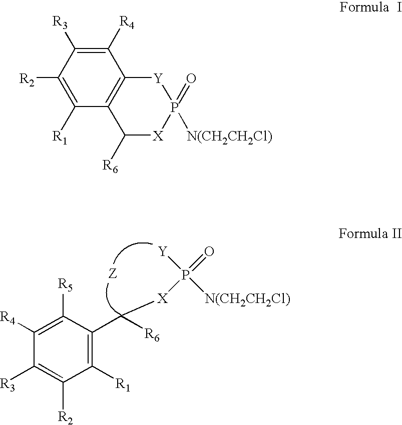
wherein at least one of R₁, R₃ or R₅ is a nitro group and the remaining substituents, R₁, R₂, R₃, R₄, and R₅, are independently a hydrogen, lower alkyl, amino, mono- or di-alkyl amino, alkanoyl amino, hydroxy, alkoxy, alkoxycarbonyl, carbamoyl, cyano, formyl, carboxyl or halogen group;
R₆ is a hydrogen, lower alkyl that is unsubstituted or substituted by free or alkylated amino, piperazinyl, piperidyl, pyrrolidinyl or morpholinyl, hydroxy, alkoxy, alkoxycarbonyl, carbamoyl, carboxyl or cyano group;
X and Y are each independently O, NH, NCH₂CH₂Cl or N(CH₂CH₂Cl)₂;
Z is two separate hydrogens or a methylene, ethylene, or propylene that is unsubstituted or substituted by free or alkylated amino, piperazinyl, piperidyl, pyrrolidinyl or morpholinyl, hydroxy, alkoxy, alkoxycarbonyl, carbamoyl, or cyano.

2. A method of producing a nitroaryl-substituted phosphoramide of claim 1 comprising condensing a precursor alcohol, amino alcohol, diamine, or diol with bis(2-chloroethyl)phosphoramidic dichloride thereby producing a nitroaryl-substituted phosphoramide.

3. A pharmaceutical composition comprising a compound of claim 1 and a pharmaceutically acceptable carrier.

4. A method for inhibiting undesirable cell growth or proliferation comprising administering an effective amount of a pharmaceutical composition of claim 3 so that undesirable cell growth or proliferation is inhibited.

5. The method of claim 4 further comprising administering a reducing agent with the pharmaceutical composition.
